(19) United States
(12) Patent Application Publication
(10) Pub. No.: US 2016/0136287 A1
(43) Pub. Date: May 19, 2016

(54) CONJUGATED VACCINE

(71) Applicant: The University of Birmingham, Birmingham (GB)

(72) Inventors: Kai-Michael Toellner, Birmingham (GB); Roy Bicknell, Birmingham (GB)

(73) Assignee: The University of Birmingham, Birmingham (GB)

(21) Appl. No.: 14/924,219

(22) Filed: Oct. 27, 2015

(30) Foreign Application Priority Data
Oct. 27, 2014 (GB) 1419061.5

(51) Int. Cl. A61K 47/48; A61K 39/00

(52) U.S. Cl. CPC A61K 47/4833; A61K 39/0005; A61K 2039/6037; A61K 2039/6031

(57) ABSTRACT

The present invention provides a composition for provoking an immune response in a patient to an autoantigen target, the composition comprising the target conjugated a carrier polypeptide.

The present invention relates to a composition for provoking an immune response in a patient to an autoantigen target.

### INTRODUCTION

Although improvements to cancer therapies over recent years have led to reduced age adjusted cancer mortalities, total numbers of cancer deaths are still growing, partly due to population growth, but mainly because of the increase in age of Western populations. New and cost-efficient therapies are clearly needed. Several immunotherapies, based either on monoclonal antibodies specific for cancer expressed antigens, or active vaccination inducing T cell immunity to cancer related proteins, are being developed or are currently being tested in the clinic. As T cell immunity is dependent on activation through patient specific antigen-presenting cells, these are often custom produced. Further targets, especially targets broadly expressed and widely usable for a large range of tumours, are needed. Indeed, further vaccines with broad applicability to a range of cancers across a population are needed.

Autoimmune diseases involve autoantigens and are also a growing problem. Examples include Lupus erythematosus, Sjögren's syndrome, scleroderma, rheumatoid arthritis, and dermatomyositis.

Surprisingly, we have found that a simple conjugated vaccine can be provided that provides a rapid vaccination to a patient or individual to an autoantigen.

### SUMMARY OF THE INVENTION

Thus, in a first aspect the invention provides a composition for provoking an immune memory response in a patient to an autoantigen target, the composition comprising the target conjugated to a carrier polypeptide, against which immunological memory exists in the patient. In one embodiment, the composition consists of the target conjugated to a carrier polypeptide, against which immunological memory exists in the patient.

All that is required is that the target is conjugated to a carrier protein, and that the carrier protein will itself elicit an immune memory response in the patient. As such, the patient will have been exposed to the carrier protein, or elements thereof, before the present composition is administered. The pre-exposure is typically at least several weeks or even 1 or 2 months in advance of the administration of the present composition, such that immune memory has been generated to at least one epitope comprised on or within the carrier protein. Upon re-administration of the carrier protein, this time conjugated to the target, the immune memory response to the carrier is again raised (elicited). Optionally, therefore, we recruit T-cell memory against the carrier. This is harnessed to achieve rapid and efficient activation of B cell activity. Optionally, the antigen will also be bound by residual antibodies specific to the carrier. This may help to activate the vaccine by opsonization.

Diphtheria toxoid or Tetanus toxoid, e.g. the non-toxic fragment C of tetanus toxin (FrC) (in some aspects of this invention these terms may be used interchangeably) are examples of carrier proteins against which the patient has (or is at least very likely to have) an immune memory response. Indeed, Diphtheria toxoid is known to be used in conjugated vaccines, but only when conjugated to bacterial polysaccharide (Schneerson et al., 1986). Instead, we conjugate our carrier protein, for instance Diphtheria toxoid, to an autoantigen target. Bacterial polysaccharides are not autoantigen targets.

Conversely, some companies such as BiovaxlD provide personalised cancer vaccines by conjugating an autoantigen from a specific patient, but the carrier protein used does not elicit an immune memory response in the patient (at the time of administering the conjugate). In other words, in this cancer system, no immunological memory exists in the patient against the carrier.

The composition is, optionally, a liquid. This may be for parenteral administration, e.g. intramuscular application. Other forms of administration may include transdermal patches.

The immune memory response may be provoked, elicited or raised, the terms can be used interchangeably herein. This occurs in the patient and consists of an immune memory response against the carrier protein or a fragment thereof, i.e. against an antigenic portion of the carrier. This antigenic portion is recognised by the patient's immune system and a memory response against that antigenic portion of the carrier is initiated. This typically consists of a response of T memory and/or B memory cells and/or antibody specific for the carrier protein or fragments thereof.

There may, of course, be more than one antigenic portion on (or within) the carrier that is recognised by the patient's immune memory, the only requirement in this regard is that there is at least one.

The carrier may be a polypeptide or a protein, the terms can be used interchangeably herein. Typically, the carrier comprises at least 10 amino acids.

Optionally, the carrier may comprise or consist of an antigen commonly used in human vaccination, particularly common vaccination programs that are implemented in the vast majority of the population. These may include one or more of the antigens used in the polio vaccine. Other alternatives include measles, mumps, rubella, HPV and pertussis components. In particular, for instance, the majority of the population is immune to Diphtheria and Tetanus toxoid, having been vaccinated against it at an early age. Indeed, the Diphtheria toxoid is already used in conjugate vaccination. It is used in adults and infants (Eskola et al., 1987) to induce long-lasting high affinity immunity to antigens that cannot be presented to T cells because they are not processed by antigen-presenting cells: T-independent polysaccharide antigens expressed by encapsulated bacteria. In other words, Diphtheria toxoid has been safely tested and proved effective in conjugate vaccines when linked to bacterial polysaccharides.

Advantages of using Diphtheria or Tetanus toxoid (also called the non-toxic fragment C of tetanus toxin (FrC)) are that they are defined small polypeptides, available purified in large amounts and that vectors for genetic coupling exist.

The amino acid sequence of Diptheria toxoid is provided as SEQ ID NO: 1

| | |
|---|---|
| 1 | gaddvvdssk sfvmenfssy hgtkpgyvds iqkgiqkpks gtqgnyddqw kgfystdnky |
| 61 | daagysvdne nplsgkaggv vkvtypgltk vlalkvdnae tikkelglsl teplmeqvgt |
| 121 | eefikrfgdg asrvvlslpf aegsssveyi nnweqakals veleinfetr gkrgqdamve |
| 181 | ymaqacagnr vrrsvgssls cinldwdvir dktktkiesl kehgpiknkm sespnktvse |
| 241 | ekakqyleef hqtalehpel selktvtgtn pvfaganyaa wavnvaqvid setadnlekt |
| 301 | taalsilpgi gsvmgiadga vhhnteeiva qsialsslmv aqaiplvgel vdigfaaynf |
| 361 | vesiinlfqv vhnsynrpay spghkt |

The amino acid sequence of FrC is provided as SEQ ID NO: 2

| | |
|---|---|
| 1 | mknldcwvdn eedidvilkk stilnldinn diisdisgfn ssvitypdaq lvpgingkai |
| 61 | hlvnnessev ivhkamdiey ndmfnnftvs fwlrvpkvsa shleqygtne ysiissmkkh |
| 121 | slsigsgwsv slkgnnliwt lkdsagevrq itfrdlpdkf naylankwvf ititndrlss |
| 181 | anlyingvlm gsaeitglga irednnitlk ldrcnnnnqy vsidkfrifc kalnpkeiek |
| 241 | lytsylsitf lrdfwgnplr ydteyylipv assskdvqlk nitdymyltn apsytngkln |
| 301 | iyyrrlyngl kfiikrytpn neidsfvksg dfiklyvsyn nnehivgypk dgnafnnldr |
| 361 | ilrvgynapg iplykkmeav klrdlktysv qlklyddkna slglvgthng qigndpnrdi |
| 421 | liasnwyfnh lkdkilgcdw yfvptdegwt nd |

In one embodiment therefore, the carrier protein may be selected from at least SEQ ID NO: 1 or 2 or a biologically active fragment or variant thereof. A biologically active variant of SEQ ID NO: 1 or 2 may differ from these sequences by as few as 1-15 amino acid residues, as few as 1-10, such as 6-10, as few as 5, as few as 4, 3, 2, or even 1 amino acid residue. In certain embodiments, SEQ ID NO: 1 or 2 may be altered in various ways including amino acid substitutions, deletions, truncations, and insertions. Methods for such manipulations are generally known in the art. For example, amino acid sequence variants and fragments of SEQ ID NOs 1 or 2 can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. The deletions, insertions, and substitutions of the protein sequences encompassed herein are not expected to produce radical changes in the characteristics of the protein. When it is difficult, however, to predict the exact effect of a substitution, deletion, or insertion in advance of making such modifications, one skilled in the art will appreciate that the effect will be evaluated by routine screening assays. In one embodiment, the variant has at least 75% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the sequence represented by SEQ ID NO: 1 or 2.

A “fragment” means a portion of the amino acid sequence and hence a portion of the protein encoded thereby. In one embodiment, the fragment is a T-cell epitope, that is, the fragment is of a length sufficient to elicit a T-cell response. In one embodiment, the fragment is between 8 and 11 amino acids in length. In another embodiment, the fragment is between 12 and 17 or 13 and 17 amino acids in length. Such fragments may be readily prepared, for example, by chemical synthesis of the fragment by application of nucleic acid amplification technology or by introducing selected sequences into recombinant vectors for recombinant production.

Coupling the autoantigen to a live virus is possible, but may also be excluded.

One option is, therefore, to link the autoantigen to Diphtheria Toxoid or non-toxic fragment C of tetanus toxin (FrC) as the carrier.

Indeed, linkage of the autoantigen to one or more carriers is envisaged to widen the chances that the patient will respond to the composition. As such, the autoantigen may be linked to two or more carriers, at least one of which is the Diphtheria or the Tetanus toxoid. The autoantigen may also be linked to both of said toxoids. The patient may also be treated consecutively with autoantigen coupled to various carriers. This may increase the chances of a response. It may also benefit patients having developed T cell tolerance to carriers used at an earlier stage.

In one embodiment, conjugation of the target to the carrier may be either chemically or through genetic engineering (i.e. in the form of a recombinant protein, for instance a fusion protein or via an encoded linker). The conjugation is typically through covalent bonding rather than electrostatic interactions, for instance. Suitable linkers may be used to link the carrier to the target. Another way of looking at this is that the linker separates the carrier from the target. Conjugation in this sense may also be considered to be coupling. We exemplify herein the use of the Fc part of human immunoglobulin. Whilst this serves to prove the point, it is generally thought that this would not be a good carrier for vaccination of humans.

It will be appreciated that there may be some steric factors to consider, such as whether the conjugation site or linker obscures the carrier antigenic portion. This may be helpful in some circumstances, if it aids in slowing immune clearance perhaps, but in general this is to be avoided.

The target is an autoantigen. An autoantigen is typically an antigen that, whilst being a normal constituent of the patient's body, is nevertheless the subject of a humoral or cell-mediated immune response (i.e. a humoral or cell-mediated immune response is directed against that autoantigen).

Optionally, the autoantigen target may be the target of an autoimmune response triggered by autoimmune disease. The autoimmune target may be CD20 or TNF-alpha, i.e. any protein or other structure, e.g. glycosylations against which an immune response can be raised and ideally this should assist in the prophylaxis or treatment of the autoimmune condition.

Optionally, the target is a cancer target, i.e. a cancer target against which it is desired to raise a humoral or cell-mediated immune response. Suitable examples include Robo4, Clec14a, EGFR, Her2, CD38, CD52 or VEGF. The cancer target may be considered to be a cancer-related target. Vascular surface expressed tumour antigens are also preferred. Most typically, the cancer target will be a protein or polypeptide.

The target may be a native autoantigen or an altered (i.e. mutated) version of an autoantigen. For instance, some of the present cancer targets are themselves mutated/altered versions of autoantigens. Other cancer targets may not, however, be altered versions of autoantigens.

T cell immunity to the carrier protein may be induced at an early stage by vaccination with the carrier protein in alum adjuvant, generating T cell memory that will be immediately available upon further vaccination.

A single target is preferred, as opposed to a mixture of different targets. The target is also preferably defined, i.e. is a specific protein for instance, thus distinguishing the present target from mixtures and especially random mixtures such as cell lysates. In this sense, the target may be considered to be pre-defined, i.e. defined in advance. Optionally, therefore, Tumour Cell Lysate (TCL) is excluded. TCL is a mixture of poorly defined antigens which vary greatly between samples from the same individual, let alone multiple individuals.

The target may be endogenous to the individual. It may have been sampled from the patient to be treated by the present composition, in which case a companion diagnostic may be included with the present composition in a kit. Thus, also provided is a kit comprising the composition as defined herein and a companion diagnostic for a disease condition to be treated in a patient. In a further embodiment, the kit further comprises instructions for use. Alternatively, it may be a commonly found target, which may be used in public vaccination strategies. Optionally, the target is present in the majority of the population to be administered to and even various forms may be envisaged.

The target is associated with a disease condition. Autoimmune targets are associated with autoimmune disease, whilst cancer targets are associated with cancerous conditions. In some cases, the target may be associated with one or more autoimmune diseases. In other cases, the target may be associated with one or more cancerous conditions. If any cancer is considered to be autoimmune, then the present conditions may include cancer or a non-cancerous autoimmune disease.

Preferred are autoimmune and inflammatory diseases, especially where monoclonal antibodies, e.g. antibodies specific for TNF alpha, α4β7 integrin, BAFF, CD2, CD3, CD20, CD22, CD80, CD86, C5 complement, IgE, IL-113, IL-5, IL-6R, IL-12, IL23, are administered. Autoimmune diseases involving autoantigens may include systemic lupus erythematosus, Sjögren's syndrome, scleroderma, rheumatoid arthritis, dermatomyositis, multiple sclerosis, Crohn's disease, psoriasis, psoriatic arthritis, ulcerative colitis, ankylosing spondylitis.

The cancer to be treated or vaccinated against (i.e. the, or one of the, cancerous condition(s) associated with the cancer target) may be bladder cancer, pancreatic cancer, Lung cancers, e.g. Lewis lung carcinoma or any other cancer expressing specific autoantigens.

It will be appreciated that where a condition is mentioned, the target is one that is associated with that condition, and visa versa. Thus, if the condition to be treated is Lupus, then the target is chosen from Lupus autoantigens. If the condition to be treated is prostate cancer, then the target is an autoantigen associated with prostate cancer.

Treatment and prophylaxis can be interchangeably herein. In the present invention, prophylaxis of a condition includes vaccination thereagainst.

The target used in the present composition may be a fragment or variant of the full protein against which activity is sought. Both “fragment” and “variant” are defined elsewhere herein. It may be only a small fragment, say of 10 amino acids, but must be sufficiently sized to invoke the required immune memory response against it.

In one embodiment, the autoantigen is Robo4 or Clec14a or a variant or fragment thereof. Robo4 is described in the Bicknell PCT mentioned below. One especially preferred option is therefore to provide Robo4 or Clec14a conjugated to the Diphtheria toxoid.

The amino acid sequence of Clec14a is provided as SEQ ID NO: 3:

| | |
|---|---|
| 1 | mrpafalcll wqalwpgpgg gehptadrag csasgacysl hhatmkrqaa eeacilrgga |
| 61 | lstvragael ravlallrag pgpgggskdl lfwvalerrr shctlenepl rgfswlssdp |
| 121 | gglesdtlqw veepqrscta rrcavlqatg gvepagwkem rchlrangyl ckyqfevlcp |
| 181 | aprpgaasnl syrapfqlhs aaldfsppgt evsalcrgql pisvtciade igarwdklsg |
| 241 | dvlcpcpgry lragkcaelp nclddlggfa cecatgfelg kdgrscvtsg eggptlggtg |
| 301 | vptrrppata tspvpqrtwp irvdeklget plvpeqdnsv tsipeiprwg sqstmstlqm |
| 361 | slqaeskati tpsgsviskf nsttssatpq afdsssavvf ifvstavvvl viltmtvlgl |
| 421 | vklcfhesps sqprkesmgp pglesdpepa algsssahct nngvkvgdcd lrdraegall |
| 481 | aesplgssda |

The amino acid sequence of the first isoform of Robo4 is provided as SEQ ID NO: 4: (NCBI Reference Sequence: NP_061928.4)

```
                10 20 30 40
MGSGGDSLLG GRGSLPLLLL LIMGGMAQDS PPQILVHPQD
                50 60 70 80
QLFQGPGPAR MSCQASGQPP PTIRWLLNGQ PLSMVPPDPH
                90 100 110 120
HLLPDGTLLL LQPPARGHAH DGQALSTDLG VYTCEASNRL
              130 140 150 160
GTAVSRGARL SVAVLREDFQ IQPRDMVAVV GEQFTLECGP
              170 180 190 200
PWGHPEPTVS WWKDGKPLAL QPGRHTVSGG SLLMARAEKS
              210 220 230 240
DEGTYMCVAT NSAGHRESRA ARVSIQEPQD YTEPVELLAV
              250 260 270 280
RIQLENVTLL NPDPAEGPKP RPAVWLSWKV SGPAAPAQSY
              290 300 310 320
TALFRTQTAP GGQGAPWAEE LLAGWQSAEL GGLHWGQDYE
              330 340 350 360
FKVRPSSGRA RGPDSNVLLL RLPEKVPSAP PQEVTLKPGN
              370 380 390 400
GTVFVSWVPP PAENHNGIIR GYQVWSLGNT SLPPANWTVV
              410 420 430 440
GEQTQLEIAT HMPGSYCVQV AAVTGAGAGE PSRPVCLLLE
              450 460 470 480
QAMERATQEP SEHGPWTLEQ LRATLKRPEV IATCGVALWL
              490 500 510 520
LLLGTAVCIH RRRRARVHLG PGLYRYTSED AILKHRMDHS
              530 540 550 560
DSQWLADTWR STSGSRDLSS SSSLSSRLGA DARDPLDCRR
              570 580 590 600
SLLSWDSRSP GVPLLPDTST FYGSLIAELP SSTPARPSPQ
              610 620 630 640
VPAVRRLPPQ LAQLSSPCSS SDSLCSRRGL SSPRLSLAPA
              650 660 670 680
EAWKAKKKQE LQHANSSPLL RGSHSLELRA CELGNRGSKN
              690 700 710 720
LSQSPGAVPQ ALVAWRALGP KLLSSSNELV TRHLPPAPLF
              730 740 750 760
PHETPPTQSQ QTQPPVAPQA PSSILLPAAP IPILSPCSPP
              770 780 790 800
SPQASSLSGP SPASSRLSSS SLSSLGEDQD SVLTPEEVAL
              810 820 830 840
CLELSEGEET PRNSVSPMPR APSPPTTYGY ISVPTASEFT
              850 860 870 880
DMGRTGGGVG PKGGVLLCPP RPCLTPTPSE GSLANGWGSA
              890 900 910 920
SEDNAASARA SLVSSSDGSF LADAHFARAL AVAVDSFGFG
              930 940 950 960
LEPREADCVF IDASSPPSPR DEIFLTPNLS LPLWEWRPDW
              970 980 990 1000
LEDMEVSHTQ RLGRGMPPWP PDSQISSQRS QLHCRMPKAG
ASPVDYS
```

Another isoform of ROBO4 is also known and may be used in place of that given in SEQ ID NO: 4.

SEQ ID NO: 5: Robo4 isoform 2 (NCBI Reference Sequence: NP_001288017.1):

| | |
|---|---|
| 1 | mvavvgeqft lecgppwghp eptvswwkdg kplalqpgrh tvsggsllma raeksdegty |
| 61 | mcvatnsagh resraarvsi gepqdytepv ellavriqle nvtllnpdpa egpkprpavw |
| 121 | lswkvsgpaa paqsytalfr tqtapggqga pwaeellagw qsaelgglhw gqdyefkvrp |
| 181 | ssgrargpds nvlllrlpek vpsappqevt lkpgngtvfv swvpppaenh ngiirgyqvw |
| 241 | slgntslppa nwtvvgeqtq leiathmpgs ycvqvaavtg agagepsrpv cllleqamer |
| 301 | atqepsehgp wtleqlratl krpeviatcg valwllllgt avcihrrrra rvhlgpglyr |
| 361 | ytsedailkh rmdhsdsqwl adtwrstsgs rdlsssssls srlgadardp ldcrrsllsw |
| 421 | dsrspgvpll pdtstfygsl iaelpsstpa rpspqvpavr rlppqlaqls spcsssdslc |
| 481 | srrglssprl slapaeawka kkkqelqhan sspllrgshs lelracelgn rgsknlsqsp |
| 541 | gavpqalvaw ralgpkllss snelvtrhlp paplfphetp ptqsqqtqpp vapqapssil |
| 601 | lpaapipils pcsppspqas slsgpspass rlsssslssl gedqdsvltp eevalclels |
| 661 | egeetprnsv spmprapspp ttygyisvpt aseftdmgrt gggvgpkggv llcpprpclt |
| 721 | ptpsegslan gwgsasedna asaraslvss sdgsfladah faralavavd sfgfglepre |
| 781 | adcvfidass ppsprdeifl tpnlslplwe wrpdwledme vshtqrlgrg mppwppdsqi |
| 841 | ssqrsqlhcr mpkagaspvd ys |

An antigenic portion of carrier, e.g. Diphtheria toxoid, must be used. Similarly, sufficient (but not necessarily all) of the target must also be provided in order to provoke the required immune response. This can be assessed on a simple trial and error basis or from what is already known about the target and the immunogenic portions thereof. In one embodiment, the whole of the target may be provided, in the sense of the full sequence or at least that normally encountered by the patient outside of this vaccination, as it will be appreciated that the intention is to vaccinate the patient against forms of the target that they would commonly be exposed to (which may include some post-translational modification etc.).

Polynucleotides encoding these Robo4 and Clec14a amino acids are known and may, in any case, be derived from the above sequence.

Optionally, T cell memory may be recruited. This is most preferably recruited for cancer targets. Indeed, it may be that only T cell memory is recruited, with little or no antibody response to the target.

The composition may, optionally, include no adjuvant. In particular, a separate adjuvant is preferred. In some cases it is known to use the M2 adjuvant, but this is also preferably excluded.

Also provided is a method of vaccinating an individual comprising administering the present composition to a patient to thereby elicit the immune response to said target in said patient. In addition, there is provided a vaccine and use thereof for preventing cancer or an autoimmune disorder, wherein the vaccine comprises a composition as defined herein and optionally an adjuvant.

Also provided is the use of a composition as defined herein to provoke an immune memory response in a patient to an autoantigen.

A booster is also provided, as is a method of boosting a vaccinated individual. Individuals may be immunised repeatedly to maintain and/or boost autoantigen-specific antibody levels, B cell and plasma cell numbers and/or T cell numbers and to increase the affinity of the autoantigen-specific antibody. Provided is, therefore, a method of vaccinating an individual and/or boosting a vaccinated individual comprising administering the present composition to a patient to thereby elicit the immune response to said target in said patient.

The patient has been exposed to the carrier, or a fragment thereof, previously. Typical, therefore, the patient is immune to the carrier. The carrier is capable of eliciting an immune response when administered as the conjugate. This may be the first time that the conjugate is administered and is certainly the first time that the target has been exposed in the presence of the carrier.

Also provided is a method for the prophylaxis or treatment of an autoimmune disease by administering to the patient in need thereof the carrier with an autoantigen from that disease. Similarly, the invention also provides a method for the prophylaxis or treatment of cancer by administering to the patient in need thereof the carrier with an autoantigen from that disease. ‘Treatment’ refers to the management of a patient through medical or surgical means. The treatment improves or alleviates at least one symptom of a medical condition or disease and is not required to provide a cure. In one embodiment, the cancer may be selected from bladder cancer, pancreatic cancer, Lung cancers, or any other cancer expressing specific autoantigens. In another embodiment, the autoimmune disorder may be selected from systemic lupus erythematosus, Sjögren's syndrome, scleroderma, rheumatoid arthritis, dermatomyositis, multiple sclerosis, Crohn's disease, psoriasis, psoriatic arthritis, ulcerative colitis, ankylosing spondylitis.

The composition may be described as a conjugate vaccine, comprising the above elements.

An example of chemical conjugation is provided in Garside, P., et al, 1998. Garside describe an example of chemical coupling. The response studied is not carrier-primed, but can be used according to our invention nonetheless. They describe a method of Immunization using Hen egg lysozyme (HEL) to chicken ovalbumine (cOVA), producing HEL-cOVA conjugate. We have performed similar conjugations in our lab using mouse Robo4-Fc and chicken gamma globulin (CGG): Purified mouse Robo4-Fc protein was cross-linked to CGG using glutaraldehyde. Accordingly, in one embodiment, glutaraldehyde is used to chemically conjugate the target to the carrier polypeptide. In brief, 2 μl of glutaraldehyde 25% stock (Sigma, Gillingham, UK) was added to 1 ml of reaction mix containing 1 mg of mouse Robo4-Fc protein and 1 mg of CGG in PBS (pH 7.5-8). The human Fc protein alone was also CGG crosslinked following an identical procedure. The reaction mix was incubated at room temperature (RT) for 10 min. The reaction was quenched by adding 100 μl of 1 M Tris-HCl (pH 8) and left at RT for 15 min. Before injecting into mice, the mix was dialysed (10,000 MWCO) with PBS overnight. 50 μg of Robo4-CGG or Fc-CGG conjugate was subcutaneously injected into the 5-week CGG primed mice. Simultaneously, each mouse was received 10⁶ Lewis lung carcinoma cell subcutaneously. Similar methods may be used to chemically conjugate the target to the carrier in the present invention.

Alternatively, a recombinant technique may be used. Two examples of this are the genetic engineering of Robo4 with human Fc described in FASEB J. 2005 January; 19(1):121-3. Epub 2004 Oct. 14, and the methods described in “Soluble Robo4 receptor inhibits in vivo angiogenesis and endothelial cell migration” by Suchting S, Heal P, Tahtis K, Stewart L M and Bicknell R.

In another aspect the invention relates to a vector comprising a nucleic acid encoding a target and a nucleic acid encoding a carrier polypeptide. In one embodiment the target is CLEC14A and/or the carrier polypeptide is FrC. In one embodiment, the vector comprises at least one nucleic acid as defined herein. In one embodiment, the vector comprises a nucleic acid as defined in SEQ ID NO: 13 and/or SEQ ID NO: 16. In another embodiment, the vector comprises a nucleic acid as defined in SEQ ID NO: 21. The vector is preferably an expression vector. A suitable expression vector would be well known to the skilled person. The vector may further comprise a regulatory sequence that directs expression of the nucleic acid. Again, a suitable regulatory sequence would be well known to the skilled person. Marker genes can also be included.

In another aspect the invention relates to a host cell comprising a vector as defined above. The host cell may be a mammalian or bacterial cell. The invention also relates to a culture medium or kit comprising a culture medium and an isolated host cell as described above.

The invention is further described in the following non-limiting figures.

### BRIEF DESCRIPTION OF THE FIGURES

FIG. 1: Without being bound by theory, this figure points at the expected immunology behind the conjugate vaccine. Mice receiving initial immunization with CGG in alum were later immunized subcutaneously with Robo4-Fc-CGG (crosslinked using glutaraldehyde). Simultaneously, 10⁶ Lewis lung carcinoma cells were implanted. Tumour volume was plotted as mean±SEM. Two-way ANOVA analysis of tumour volume P<0.001, n=8 per group. a) Efficient induction of Robo4-specific antibodies after Robo4 autoantigen-CGG carrier protein conjugate vaccination in mice. b) Efficient inhibition of tumour growth in mice after Robo4-CGG conjugate vaccination.

FIG. 2: Inducing antibody responses to autoantigens using conjugate vaccine. a) Vaccination with autoantigen (R) will lead to activation and antigen uptake by antigen-specific B cells (B_{R}). Autoantigen-specific T cells (T_{R}) are not available, as autoantigen-specific T cells are deleted during T cell development. Activation of antigen-specific B cells leads to B cell deletion. b) Vaccination with autoantigen conjugated to a foreign protein (D) that had been used in an earlier immunisation. This leads to B cell activation, and uptake of RD. Activated B cells recruit help from D-specific memory T cells (mT_{D}) by presenting peptides of foreign protein D. This leads to rapid full activation of R-specific B cells, plasma cell differentiation (P_{R}) and R-specific antibodies.

FIG. 3: Genetic linking with CLEC14A with non-toxic fragment C of tetanus toxin.

A: Lentiviral expression vector. Human or mouse CLEC14a was linked to non-toxic fragment C of tetanus toxin (FrC). Stably transfected cells were enriched by FACS sorting for GFP expression. Sequence was confirmed for both constructs by DNA sequencing.

B: Linking mouse CLEC14a and FrC by PCR. Left amplified FrC DNA (1,388 bp), middle amplified muCLEC14a DNA (1,223 bp) and right linked muCLEC14a-FrC DNA (2,590 bp). DNA was ligated into the lentiviral expression vector and transfected into HEK293 cells. DNA sequencing confirmed the correct sequence.

C: GFP Expression in HEK293 cells. HEK 293 cells before transfection and after transfection with muCLEC14a-FrC and enrichment.

FIG. 4: CLEC14-specific blood antibody response.

Mice were unprimed (PBS control) or primed with carrier FrC in alum to induce immunological memory to the carrier. Three wk later all mice were challenged i.p. with soluble murine CLEC14a-FrC. Pre-immunisation, pre-challenge, and 5 d post challenge titres from PBS primed (open circles) and FrC primed (closed circles) mice. Colours identify individual mice. Challenge with soluble CLEC14a-FrC induces CLEC14a-specific IgG1 at 1000× above background levels, with little production of other IgG subclasses or IgM. Specific IgA or IgE were not detectable (not shown).

FIG. 5: Survival curves of mice implanted with Lewis lung carcinoma and vaccinated with muCLEC14-FrC. Pilot experiment using vaccination with chemically crosslinked muCLEC14-FrC, showing that mice primed with FrC have better survival after tumour implantation and vaccination (blue) than mice non-primed, non-vaccinated (brown).

FIG. 6: CD31 as a marker for vessels in tumour tissue investigated using immunofluorescence staining.

(A) A representative picture from each group shows the staining CD31+ vessels (green) and C1q (red) deposition with a DAPI (blue) counterstain.

(B) Percentage of area covered by CD31 quantified from the immunofluorescence stains using Fiji software. Significant difference (P=0.0155) using Mann-Whitney test (2-tailed).

### DETAILED DESCRIPTION

The present invention will now be further described. In the following passages, different aspects of the invention are defined in more detail. Each aspect may be combined with any other aspect or aspects unless clearly indicated to the contrary. In particular, any feature indicated as being preferred or advantageous may be combined with any other feature or features indicated as being preferred or advantageous.

The inventors have found that conjugating the self-protein Robo4 to soluble, antigenic fragments such as Fc and cross linking to another Ag (chicken gamma globulin) induces a protective effect and reduces tumour angiogenesis. Linkage to Ag encountered through childhood vaccination, eg Diptheria Toxoid, is also envisaged. Robo4 is linked to pancreatic, bladder, lung and prostate cancer, so it is plausible that any of these cancers can be treated (treatment or prophylaxis) by the present invention.

Ze-Yu Wang et al (Chinese Journal of Cancer, 2012, Vol. 31, issue 6, pp 295-305) uses Dip Toxin and a TCL system (a random mixture of a large number of potential antigens which varies between individuals) and no previous immunity to the carrier is described or possible, i.e. they do not seek to recruit T cell memory against a carrier. In their system, an adjuvant has to be present. In fact, their system is not suitable for human use as the Dip Toxin is used together with the M2 adjuvant, neither of which are suitable for human use.

Bicknell describes Robo4 and the targeting thereof in WO 2009/044158 (Cancer Research Technology Limited).

Accordingly, we have developed a vaccine that targets antigens widely expressed in vessels of tumour tissues: the lower shear stress in tumour vessels compared to vessels of normal tissues leads to strong expression of the tumour endothelial cell antigens Robo4 and Clec14a in a wide range of different tumours (Heath and Bicknell, 2009; Mura et al., 2012). While most current cancer specific vaccines have been designed to induce cytotoxic T cell responses, we decided to develop a protocol that induces a strong and reliable antibody response. This avoids problems with patient specific responsiveness to specific MHC molecules or peptides. However, due to thymic exclusion of autoreactive T cells, antibody responses are not easily induced to autoantigens. Therefore, Robo4 was linked to an unrelated carrier protein (either by chemical cross-linking or by genetic engineering). T cell immunity to the carrier protein was induced at an early stage by vaccination with the carrier protein in alum adjuvant, generating T cell memory that will be immediately available upon further vaccination. We have shown that subsequent vaccination with our conjugate vaccine (in absence of any further adjuvants) induces a rapid autoreactive anti-tumour vessel antibody response. This led to reduce tumour growth in a rapidly growing Lewis lung carcinoma model implanted into a subcutaneous sponge, even when vaccine was given at the time of tumour implantation. We have shown that the anti-tumour response is mediated mainly by IgG1 antibody. Mice deficient in B cells, or deficient only in IgG1 have tumour growth identical to non-vaccinated mice (data submitted for publication). A vaccination protocol of recruiting memory T cell help to induce autoreactive responses is novel.

Vascular surface expressed tumour antigens are preferred examples of the present target, being cancer autoantigen targets. As antibodies diffuse into tissues, this protocol should be widely usable for any cancer associated cell surface expressed autoantigen. Further, it may be useful for the treatment on non-cancer related diseases, e.g. autoimmune diseases, where monoclonal antibodies, e.g. to anti-TNF alpha, are currently used with good success.

A range of cancer specific monoclonal antibodies are currently used or tested for cancer therapy (and autoimmune diseases). Avastin (Bevacizumab) is a monoclonal antibody inhibiting vessel formation by targeting VEGF-A. Avastin is currently the world's most profitable drug. Other monoclonal antibodies have been licensed for clinical use. Production and administration of monoclonal antibodies is expensive and patients need to be treated for many weeks. A vaccine inducing endogenous antibody production would be not only cheaper for clinical use, it would also be cheaper to develop, as humanization of antibodies and large scale production of humanized antibodies are not necessary.

One of the advantages of the present composition is that it can be used to provide a rapid vaccination against the target.

While the foregoing disclosure provides a general description of the subject-matter encompassed within the scope of the present invention, including methods, as well as the best mode thereof, of using this invention, the following examples are provided to further enable those skilled in the art to practice this invention and provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification, including reference to sequence database identifiers, are incorporated herein by reference in their entirety. Unless otherwise specified, when reference to sequence database identifiers is made, the version number is 1.

“and/or” where used herein is to be taken as a specific disclosure of each of the two specified features or components with or without the other. For example, “A and/or B” is to be taken as specific disclosure of each of (i) A, (ii) B and (iii) A and B, just as if each is set out individually herein.

The invention is further described in the following non-limiting examples.

### EXAMPLES

### Example 1

### Immunization with Chemical Robo4-Fc-CGG Conjugates in Chicken Gamma Globulin CGG Primed Mice

For antigen priming, 50 μg of CGG (Sigma, UK) was delivered i.p. with alum adjuvant per mouse. Purified mouse Robo4-Fc protein was cross-linked to CGG using glutaraldehyde. In brief, 2 μl of 25% glutaraldehyde (Sigma, Gillingham, UK) was added to 1 mg of mouse Robo4-Fc protein and 1 mg of CGG in 1 ml phosphate buffered saline (PBS, pH 7.5). As a control the human Fc protein alone was also CGG crosslinked following an identical procedure. The reaction mix was incubated at room temperature (RT) for 10 min. The reaction was quenched by adding 100 μl of 1 M Tris-HCl (pH 8) and left at RT for 15 min. Before injecting into mice, the mix was dialysed (10,000 MWCO) against PBS overnight. 50 μg of Robo4-Fc-CGG or Fc-CGG conjugate was subcutaneously injected into 5-week CGG primed mice. Simultaneously, mice were subcutaneously implanted with Lewis Lung Carcinoma cells. Tumour size was measured at indicated days and tumour volume was calculated following the formula: length×width 2×0.4 (Attia and Weiss, 1966). ANOVA analysis was performed to compare tumour growth between Robo4 vaccinated and Fc immunised control mice.

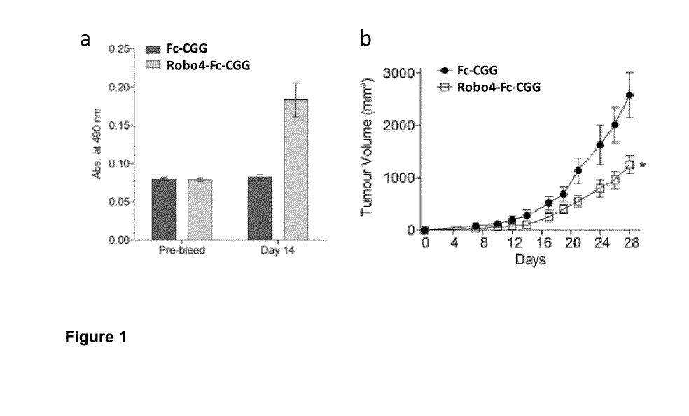

CGG immunized mice were immunized with soluble Robo4-Fc-CGG or Fc-CGG. Vaccination with Robo4-Fc-CGG led to the production of high levels of Robo4-specific IgG (FIG. 1a) and significant and sustained growth inhibition of the tumour (FIG. 1b).

### Example 2

### Immunization with Chemical Robo4-Fc-FrC Conjugates in Purified Non-Toxic Fragment C of Tetanus Toxin (FrC) Vaccinated Patients

Purified non-toxic fragment C of tetanus toxin (FrC) is cross-linked with human Robo4-Fc or Fc using glutaraldehyde. 25% glutaraldehyde is added to a mix of human Robo4-Fc or Fc protein and FrC in PBS. The reaction mixture is incubated at room temperature and then quenched by adding 100 μl of 1 M Tris-HCl (pH 8), left at room temperature for 15 min, and dialysed against PBS overnight.

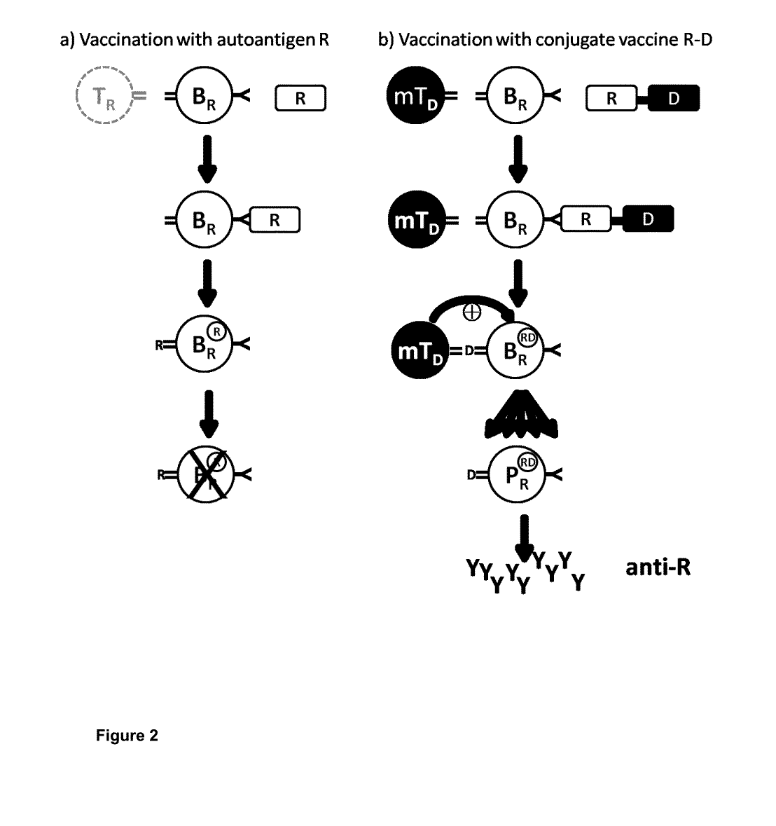

FrC vaccinated tumour patients are vaccinated with Robo4-Fc-FrC or Fc-FrC conjugate intramuscular. We expect Robo4-Fc-FrC to develop Robo4-specific antibodies within a few days. Further, we expect a specific tumour growth inhibition in the Robo4-Fc-FrC vaccinated group (FIG. 2).

### Example 3

### Genetic Linking CLEC14a with Non-Toxic Fragment C of Tetanus Toxin (FrC)

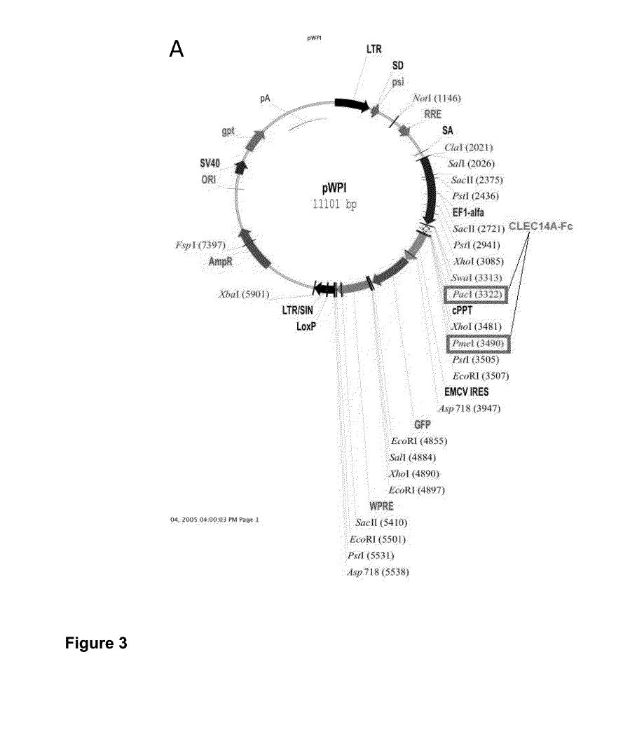

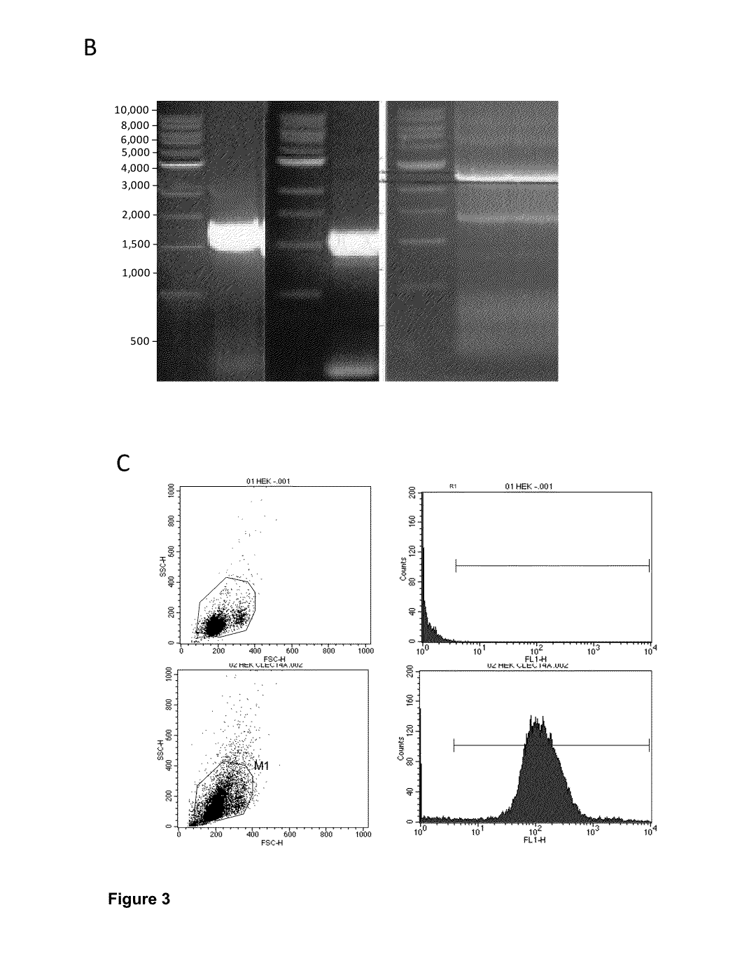

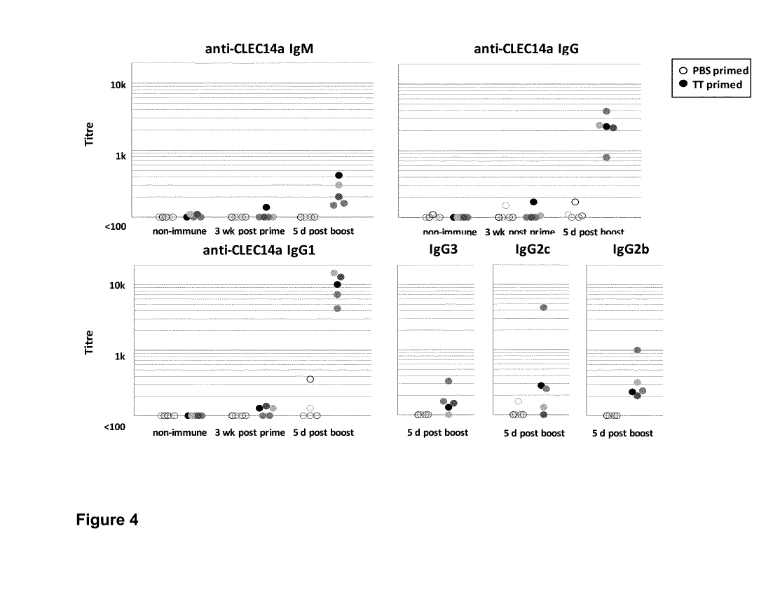

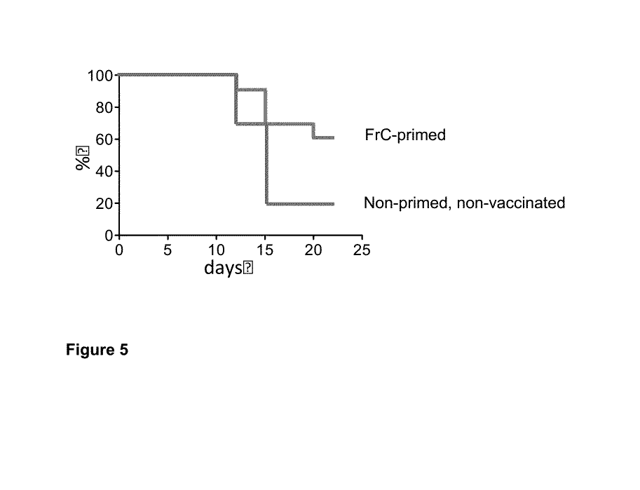

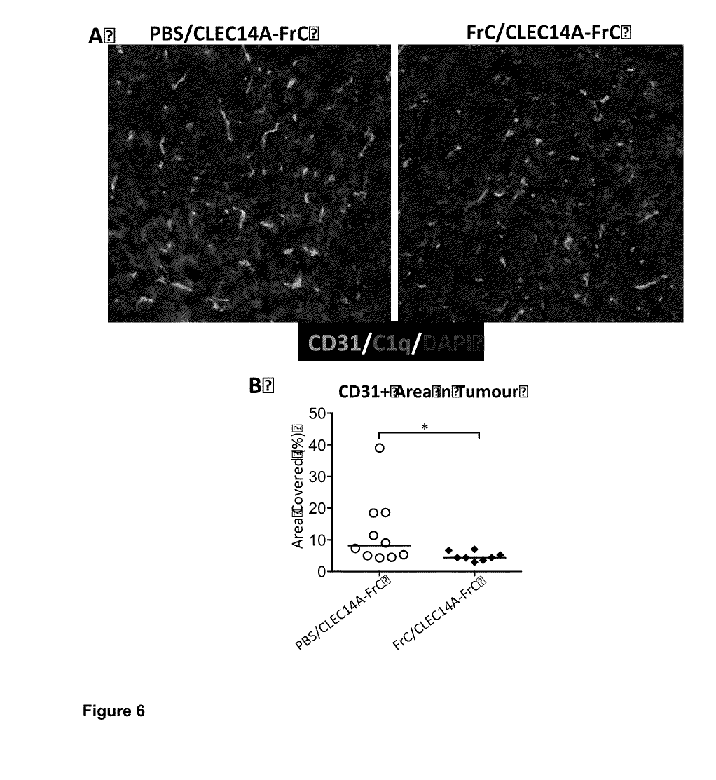

We produced vectors containing human or murine CLEC14a genetically linked to FrC. CLEC14a is widely expressed throughout different types of tumours (Mura et al., 2012). Genetic linking of huCLEC14a and muCLEC14a with FrC was achieved. The non-toxic fragment C of tetanus toxin (FrC) (plasmid pcDNA3-FrC provided by Natalia Savelyeva, Univ. Southampton) and muCLEC14a and huCLEC14a (provided by Roy Bicknell, UoB) were amplified separately by using Phusion DNA Polymerase. The muCLEC14a Forward Primer had a Pacl restriction site tail and reverse primer had an extended linker sequence. The FrC forward primer has an extended linker sequence and the reverse primer has a Pmel restriction site tail. The extended linker sequences were complementary so that the end of CLEC14a would join to the beginning of FrC with the following sequence in-between—GlyGlyGlyGlySer Linker (see Table 1). PCR products were run on a 1% agarose gel (FIG. 3B) and the bands at the expected band size were extracted by using Gene JET Gel Extraction Kit 50 preparations following the protocol. The PCR products (FrC and muCLEC14a) were linked by PCR using the CLEC14a forward primer and FrC reverse primer only. The Products were checked on gel (FIG. 3 B) and gel extracted bands. MuCLEC14a-FrC was digested with Pmel and Pacl and also the pWPI plasmid (the lentivirus plasmid used for lentivirus transfection FIG. 3A) were separately digested. Alkaline Phosphatase was added to the pWPI after digesting to stop the plasmid self-annealing. The muCLEC14a-FrC fragment into the pWPI plasmid was ligated using T4 DNA Ligase. 5 μl ligation product was transformed into a-Select Chemically Competent. Colonies were screened using Gene JET Plasmid Miniprep kit plus PCR amplification and digestion using Pmel and Pact. Those that appeared successful were then sequenced using Eurofins sequencing service. The technique for genetic linking huCLEC14a with FrC is exactly same.

Both FrC and muCLEC14a-FrC vectors were transfected into HEK293T cells. Vector containing cells were enriched by DNA sorting, using a GFP expression cassette as a selection marker for flow cytometric cell sorting (FIG. 3C). 70% of cells were already GFP positive before cell sorting. Supernatants were produced from transfected HEK293T cells. MuCLEC14a-FrC into supernatants has been confirmed by Western blotting. FrC from HEK293 FrC plasmid-transfected cells were eluted by the FrC affinity column. MuCLEC14a-FrC from HEK293 CLEC14A-FrC plasmid-transfected cell lysate were eluted by FrC affinity column plus monoclonal anti-CLEC14A antibody. A SDS-PAGE Coomassie stain was done to confirm absence of major contaminants.

### Example 4

### Antibody Responses to the Carrier FrC and Chemical Conjugates of muCLEC14a-FrC

Murine CLEC14a and FrC were chemically conjugated as described in Example 1. Briefly, equal parts of muCLEC14a and FrC were added together along with a 1/500 dilution of 25% stock glutaraldehyde) and let stand for 15 minutes. The reaction was stopped using 1M Tris-HCl pH 8 at a concentration of 100 μl/ml solution and left for 15 minutes. The mix was dialysed against PBS overnight. Mice were primed with PBS or with 50 μg of the carrier FrC in alum. Three weeks (21 days) later all mice were boosted i.p. with 50 μg soluble muCLEC14a-Frc. All mice were sacrificed at d5 after boost. Sera taken from mice were analyzed for FrC- and muCLEC14a-specific antibodies by ELISA.

FrC primed mice were immunized with soluble muCLEC14a-FrC. Vaccination with muCLEC14a-FrC led to the production of high levels of CLEC14a-specific IgG, particularly IgG1, within 5 days of immunisation in the absence of adjuvants (FIG. 4). As expected these high antibody titres do develop in the absence immune memory to the carrier (FIG. 4 PBS controls).

### Example 5

### Increased Survival of Mice Implanted with Lewis Lung Carcinoma and Vaccinated with muCLEC14a-FrC

The effects of immunization with the muCLEC14a-FrC conjugate vaccine on tumours was studied by implanting Lewis lung carcinoma (LLC) cells into wild type mice.

Mice were primed with PBS or 50 ug FrC in alum, 4 weeks later, and were immunized with 50 ug muCLEC14a-FrC or FrC. Simultaneously, mice were subcutaneously implanted with Lewis Lung Carcinoma cells. Tumour size was measured at indicated days (methods was same as it on Example 1). Mice were culled if tumour growth went beyond humane endpoints.

Mice were primed with PBS or FrC in alum, and then immunized with soluble muCLEC14a-FrC or only FrC. Mice primed with FrC have better survival after tumour implantation and vaccination than mice non-primed, non-vaccinated. (FIG. 5).

### Example 6

### Vessel Architecture or Abundance was Altered by Vaccination with CLEC14a-FrC

Tumour tissue was taken from mice which were primed with PBS or FrC in alum and 4 weeks later immunized with muCLEC14a-FrC (experiment was done with tissues from mice described in Example 5). Tumour sections were analyzed by quantifying vessel density, shape and orientation. Tumour sections were analyzed by immunstaining for CD31. Quantification of CD31⁺ vessel area was done by using Fiji software to test for effects on vessel density.

Significant more CD31+ vessel area was found within the tumours of mice non-primed mice comparing to primed plus vaccinated group (FIG. 6).

TABLE 1
Primer list

FrC
Forward with the GlyGlyGlyGlySer Linker:
5′- GGCGGAGGTGGCTCT ATG AAAAACCTTGATTGTTGG
GTCG- 3′:
SEQ ID NO: 6
3′- GAGTC CGAAAGCTGAGGAGGCCGCCTCCACCGAGATACTTT-
5′;
SEQ ID NO: 7
Reverse with PmeI restriction site:
5′- TAGTAG GTTTAAAC TTA GTC GTT GGT CCA ACC
TTC ATC- 3′;
SEQ ID NO: 8
muCLEC14a
Forward Primer with PacI restriction site:
5′- TAGTAG TTAATTAAACC ATGAGGCCG GCGTTCG- 3′;
SEQ ID NO: 9
Reverse Primer with GlyGlyGlyGlySer
complementary Linker:
5′- TTTCATAGAGCCACCTCCGCC GGAGGAGTCGAAAGCCTGAG-
3′;
SEQ ID NO: 10
PCR of FrC to muCLEC14a
Forward Primer of CLEC14a
5′- TAGTAGTTAATTAAACCATGAGGCCGGCGTTC G- 3′;
SEQ ID NO: 11
Reverse Primer of FrC:
5′- TAGTAGGTTTAAACTTAGTCGTTGGTCCAACCTTC ATC- 3′:
SEQ ID NO: 12

Additional Sequence Information:

The primer list of FrC and muCLEC14a
FrC:
SEQ ID NO: 13
AAGCTTGCCGCCACCATGGGTTGGAGCTGTATCATCTTCTTTCTGGTAGCAACAGCTACAGG

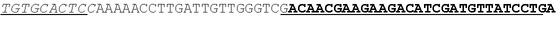

AAAAGTCTACCATTCTGAACTTGGACATCAACAACGATATTATCTCCGACATCTCTGGTTTC
AACTCCTCTGTTATCACATATCCAGATGCTCAATTGGTGCCGGGCATCAACGGCAAAGCTAT
CCACCTGGTTAACAACGAATCTTCTGAAGTTATCGTGCACAAGGCCATGGACATCGAATACA
ACGACATGTTCAACAACTTCACCGTTAGCTTCTGGCTGCGCGTTCCGAAAGTTTCTGCTTCC
CACCTGGAACAGTACGGCACTAACGAGTACTCCATCATCAGCTCTATGAAGAAACACTCCCT
GTCCATCGGCTCTGGTTGGTCTGTTTCCCTGAAGGGTAACAACCTGATCTGGACTCTGAAAG
ACTCCGCGGGCGAAGTTCGTCAGATCACTTTCCGCGACCTGCCGGACAAGTTCAACGCGTAC
CTGGCTAACAAATGGGTTTTCATCACTATCACTAACGATCGTCTGTCTTCTGCTAACCTGTA
CATCAACGGCGTTCTGATGGGCTCCGCTGAAATCACTGGTCTGGGCGCTATCCGTGAGGACA
ACAACATCACTCTTAAGCTGGACCGTTGCAACAACAACAACCAGTACGTATCCATCGACAAG
TTCCGTATCTTCTGCAAAGCACTGAACCCGAAAGAGATCGAAAAACTGTATACCAGCTACCT
GTCTATCACCTTCCTGCGTGACTTCTGGGGTAACCCGCTGCGTTACGACACCGAATATTACC
TGATCCCGGTAGCTTCTAGCTCTAAAGACGTTCAGCTGAAAAACATCACTGACTACATGTAC
CTGACCAACGCGCCGTCCTACACTAACGGTAAACTGAACATCTACTACCGACGTCTGTACAA
CGGCCTGAAATTCATCATCAAACGCTACACTCCGAACAACGAAATCGATTCTTTCGTTAAAT
CTGGTGACTTCATCAAACTGTACGTTTCTTACAACAACAACGAACACATCGTTGGTTACCCG
AAAGACGGTAACGCTTTCAACAACCTGGACAGAATTCTGCGTGTTGGTTACAACGCTCCGGG
TATCCCGCTGTACAAAAAAATGGAAGCTGTTAAACTGCGTGACCTGAAAACCTACTCTGTTC
AGCTGAAACTGTACGACGACAAAAACGCTTCTCTGGGTCTGGTTGGTACCCACAACGGTCAG
ATCGGTAACGACCCGAACCGTGACATCCTGATCGCTTCTAACTGGTACTTCAACCACCTGAA

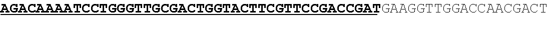

AAGCGGCCGC
Primers
Forward with the GlyGlyGlyGlySer Linker:
(SEQ ID NO: 14)

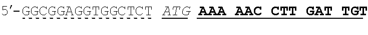

TGG GTC G-3′

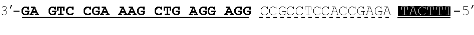

Reverse with PmeI restriction site:
(SEQ ID NO: 15)
5′-TAGTAG GTTTAAAC TTA GTC GTT GGT CCA ACC TTC ATC-3′
Annealing Temperatures
Primer 1 = 66° C.
Primer 2 = 66° C.
Annealing Temp = 69° C.
GlyGlyGlyGlySer residues for linking FrC to TEM
ATG = Start Codon
TAG = Stop Codon
Sequence of FrC
PacI restriction site for cloning into pWI vector
muCLEC14A
(SEQ ID NO: 16)
ATGAGGCCGGCGTTCGCCCTGTGCCTCCTCTGGCAGGCGCTCTGGCCCGGGCCGGGCGGCGGC
GAACACCCCACTGCCGACCGTGCTGGCTGCTCGGCCTCGGGGGCCTGCTACAGCCTGCACCAC
GCTACCATGAAGCGGCAGGCGGCCGAGGAGGCCTGCATCCTGCGAGGTGGGGCGCTCAGCACC
GTGCGTGCGGGCGCCGAGCTGCGCGCTGTGCTCGCGCTCCTGCGGGCAGGCCCAGGGCCCGGA
GGGGGCTCCAAAGACCTGCTGTTCTGGGTCGCACTGGAGCGCAGGCGTTCCCACTGCACCCTG
GAGAACGAGCCTTTGCGGGGTTTCTCCTGGCTGTCCTCCGACCCCGGCGGTCTCGAAAGCGAC
ACGCTGCAGTGGGTGGAGGAGCCCCAACGCTCCTGCACCGCGCGGAGATGCGCGGTACTCCAG
GCCACCGGTGGGGTCGAGCCCGCAGGCTGGAAGGAGATGCGATGCCACCTGCGCGCCAACGGC
TACCTGTGCAAGTACCAGTTTGAGGTCTTGTGTCCTGCGCCGCGCCCCGGGGCCGCCTCTAAC
TTGAGCTATCGCGCGCCCTTCCAGCTGCACAGCGCCGCTCTGGACTTCAGTCCACCTGGGACC
GAGGTGAGTGCGCTCTGCCGGGGACAGCTCCCGATCTCAGTTACTTGCATCGCGGACGAAATC
GGCGCTCGCTGGGACAAACTCTCGGGCGATGTGTTGTGTCCCTGCCCCGGGAGGTACCTCCGT
GCTGGCAAATGCGCAGAGCTCCCTAACTGCCTAGACGACTTGGGAGGCTTTGCCTGCGAATGT
GCTACGGGCTTCGAGCTGGGGAAGGACGGCCGCTCTTGTGTGACCAGTGGGGAAGGACAGCCG
ACCCTTGGGGGGACCGGGGTGCCCACCAGGCGCCCGCCGGCCACTGCAACCAGCCCCGTGCCG
CAGAGAACATGGCCAATCAGGGTCGACGAGAAGCTGGGAGAGACACCACTTGTCCCTGAACAA
GACAATTCAGTAACATCTATTCCTGAGATTCCTCGATGGGGATCACAGAGCACGATGTCTACC
CTTCAAATGTCCCTTCAAGCCGAGTCAAAGGCCACTATCACCCCATCAGGGAGCGTGATTTCC

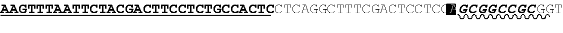

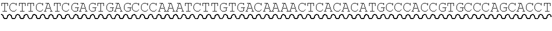

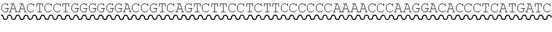

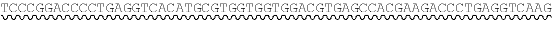

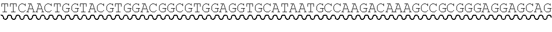

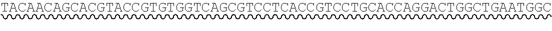

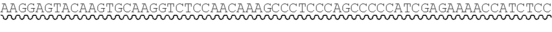

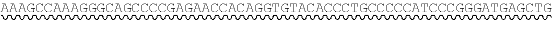

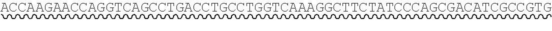

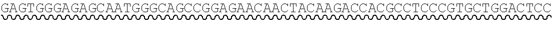

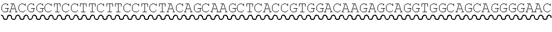

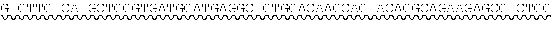

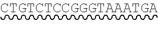

Additional base to keep in frame Restriction site NotI; Human Fc tag
Primers
Forward Primer with PacI restriction site:
(SEQ ID NO: 17)
5′-TAGTAG TTAATTAA ACC ATG AGG CCG GCG TTC G-3′
Reverse Primer with GlyGlyGlyGlySer complementary Linker:
(SEQ ID NO: 18)

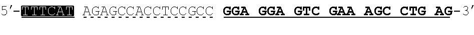

Annealing Temperatures:
Primer 1 = 64° C.
Primer 2 = 65° C.
Annealing Temp = 64° C.
6 first nucleotides of FrC
TAG = Stop Codon
PmeI restriction site for cloning into pWPI vector
Glycine residues for linking FrC to TEM
ATG = Start Codon Sequence of Clec14a extracellular domain
PCR of Frc to CLEC14A
For this I would use the reverse primer of FrC and the forward primers of CLEC14A and
ROBO4
Forward Primer of CLEC14A
(SEQ ID NO: 19)
5′-TAGTAG TTAATTAA ACC ATG AGG CCG GCG TTC G-3′
Tm Temperature-64° C.
Reverse Primer of FrC:
(SEQ ID NO: 20)
5′-TAGTAG GTTTAAAC TTA GTC GTT GGT CCA ACC TTC ATC-3′
Tm Temperature-66° C.
Sequence results
Complete Sequence of pWPI mouse Clec14a-FrC (13,450 bp)
(SEQ ID NO: 21)
TTGGAAGGGCTAATTCACTCCCAAAGAAGACAAGATATCCTTGATCTGTGGATCTACCACAC
ACAAGGCTACTTCCCTGATTAGCAGAACTACACACCAGGGCCAGGGGTCAGATATCCACTGA
CCTTTGGATGGTGCTACAAGCTAGTACCAGTTGAGCCAGATAAGGTAGAAGAGGCCAATAAA
GGAGAGAACACCAGCTTGTTACACCCTGTGAGCCTGCATGGGATGGATGACCCGGAGAGAGA
AGTGTTAGAGTGGAGGTTTGACAGCCGCCTAGCATTTCATCACGTGGCCCGAGAGCTGCATC
CGGAGTACTTCAAGAACTGCTGATATCGAGCTTGCTACAAGGGACTTTCCGCTGGGGACTTT
CCAGGGAGGCGTGGCCTGGGCGGGACTGGGGAGTGGCGAGCCCTCAGATCCTGCATATAAGC
AGCTGCTTTTTGCCTGTACTGGGTCTCTCTGGTTAGACCAGATCTGAGCCTGGGAGCTCTCT
GGCTAACTAGGGAACCCACTGCTTAAGCCTCAATAAAGCTTGCCTTGAGTGCTTCAAGTAGT
GTGTGCCCGTCTGTTGTGTGACTCTGGTAACTAGAGATCCCTCAGACCCTTTTAGTCAGTGT
GGAAAATCTCTAGCAGTGGCGCCCGAACAGGGACTTGAAAGCGAAAGGGAAACCAGAGGAGC
TCTCTCGACGCAGGACTCGGCTTGCTGAAGCGCGCACGGCAAGAGGCGAGGGGCGGCGACTG
GTGAGTACGCCAAAAATTTTGACTAGCGGAGGCTAGAAGGAGAGAGATGGGTGCGAGAGCGT
CAGTATTAAGCGGGGGAGAATTAGATCGCGATGGGAAAAAATTCGGTTAAGGCCAGGGGGAA
AGAAAAAATATAAATTAAAACATATAGTATGGGCAAGCAGGGAGCTAGAACGATTCGCAGTT
AATCCTGGCCTGTTAGAAACATCAGAAGGCTGTAGACAAATACTGGGACAGCTACAACCATC
CCTTCAGACAGGATCAGAAGAACTTAGATCATTATATAATACAGTAGCAACCCTCTATTGTG
TGCATCAAAGGATAGAGATAAAAGACACCAAGGAAGCTTTAGACAAGATAGAGGAAGAGCAA
AACAAAAGTAAGACCACCGCACAGCAAGCGGCCGCTGATCTTCAGACCTGGAGGAGGAGATA
TGAGGGACAATTGGAGAAGTGAATTATATAAATATAAAGTAGTAAAAATTGAACCATTAGGA
GTAGCACCCACCAAGGCAAAGAGAAGAGTGGTGCAGAGAGAAAAAAGAGCAGTGGGAATAGG
AGCTTTGTTCCTTGGGTTCTTGGGAGCAGCAGGAAGCACTATGGGCGCAGCGTCAATGACGC
TGACGGTACAGGCCAGACAATTATTGTCTGGTATAGTGCAGCAGCAGAACAATTTGCTGAGG
GCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCT
CTGGAAAACTCATTTGCACCACTGCTGTGCCTTGGAATGCTAGTTGGAGTAATAAATCTCTG
GAACAGATTTGGAATCACACGACCTGGATGGAGTGGGACAGAGAAATTAACAATTACACAAG
CTTAATACACTCCTTAATTGAAGAATCGCAAAACCAGCAAGAAAAGAATGAACAAGAATTAT
TGGAATTAGATAAATGGGCAAGTTTGTGGAATTGGTTTAACATAACAAATTGGCTGTGGTAT
ATAAAATTATTCATAATGATAGTAGGAGGCTTGGTAGGTTTAAGAATAGTTTTTGCTGTACT
TTCTATAGTGAATAGAGTTAGGCAGGGATATTCACCATTATCGTTTCAGACCCACCTCCCAA
CCCCGAGGGGACCCGACAGGCCCGAAGGAATAGAAGAAGAAGGTGGAGAGAGAGACAGAGAC
AGATCCATTCGATTAGTGAACGGATCTCGACGGTATCGATGTCGACGATAAGCTTTGCAAAG
ATGGATAAAGTTTTAAACAGAGAGGAATCTTTGCAGCTAATGGACCTTCTAGGTCTTGAAAG
GAGTGGGAATTGGCTCCGGTGCCCGTCAGTGGGCAGAGCGCACATCGCCCACAGTCCCCGAG
AAGTTGGGGGGAGGGGTCGGCAATTGAACCGGTGCCTAGAGAAGGTGGCGCGGGGTAAACTG
GGAAAGTGATGTCGTGTACTGGCTCCGCCTTTTTCCCGAGGGTGGGGGAGAACCGTATATAA
GTGCAGTAGTCGCCGTGAACGTTCTTTTTCGCAACGGGTTTGCCGCCAGAACACAGGTAAGT
GCCGTGTGTGGTTCCCGCGGGCCTGGCCTCTTTACGGGTTATGGCCCTTGCGTGCCTTGAAT
TACTTCCACTGGCTGCAGTACGTGATTCTTGATCCCGAGCTTCGGGTTGGAAGTGGGTGGGA
GAGTTCGAGGCCTTGCGCTTAAGGAGCCCCTTCGCCTCGTGCTTGAGTTGAGGCCTGGCCTG
GGCGCTGGGGCCGCCGCGTGCGAATCTGGTGGCACCTTCGCGCCTGTCTCGCTGCTTTCGAT
AAGTCTCTAGCCATTTAAAATTTTTGATGACCTGCTGCGACGCTTTTTTTCTGGCAAGATAG
TCTTGTAAATGCGGGCCAAGATCTGCACACTGGTATTTCGGTTTTTGGGGCCGCGGGCGGCG
ACGGGGCCCGTGCGTCCCAGCGCACATGTTCGGCGAGGCGGGGCCTGCGAGCGCGGCCACCG
AGAATCGGACGGGGGTAGTCTCAAGCTGGCCGGCCTGCTCTGGTGCCTGGCCTCGCGCCGCC
GTGTATCGCCCCGCCCTGGGCGGCAAGGCTGGCCCGGTCGGCACCAGTTGCGTGAGCGGAAA
GATGGCCGCTTCCCGGCCCTGCTGCAGGGAGCTCAAAATGGAGGACGCGGCGCTCGGGAGAG
CGGGCGGGTGAGTCACCCACACAAAGGAAAAGGGCCTTTCCGTCCTCAGCCGTCGCTTCATG
TGACTCCACGGAGTACCGGGCGCCGTCCAGGCACCTCGATTAGTTCTCGAGCTTTTGGAGTA
CGTCGTCTTTAGGTTGGGGGGAGGGGTTTTATGCGATGGAGTTTCCCCACACTGAGTGGGTG
GAGACTGAAGTTAGGCCAGCTTGGCACTTGATGTAATTCTCCTTGGAATTTGCCCTTTTTGA

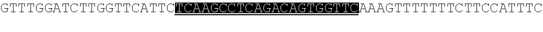

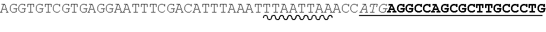

TGCCTCCTCTGTCCTGCGTTCTGGCCTCGGCCAGGGAATGGGGAGCATCCCACGGCCGATCG
CGCAGCTTGTTCGGCCTCGGGGGCTTGCTACAGCCTTCACCACGCTACCTTCAAGAGAAGGG
CGGCGGAGGAGGCCTGCAGCCTAAGGGGCGGGACTCTCAGCACCGTGCACTCAGGCTCGGAG
TTTCAAGCTGTGCTCCTGCTCTTGCGTGCAGGTCCCGGGCCTGGCGGAGGCTCCAAAGATCT
TCTGTTCTGGGTGGCTCTGGAACGCAGCATCTCACAGTGCACTCAGGAGAAAGAGCCTTTAA
GGGGTTTCTCCTGGTTGCACCCGGACTCAGAAGACTCAGAGGACAGCCCACTACCGTGGGTG
GAAGAGCCACAACGTTCCTGTACAGTGAGAAAGTGCGCTGCGCTCCAGGCCACCAGGGGAGT

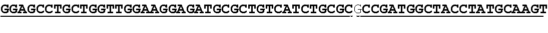

ACCAGTTTGAGGTTCTGTGCCCTGCACCTCGCCCAGGAGCCGCCTCTAATTTGAGTTTCCAA
GCTCCCTTCCGGCTGAGCAGCTCCGCGCTGGACTTCAGCCCTCCTGGGACAGAGGTGAGTGC

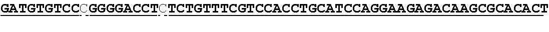

GGGACGGGCTTTTCCCTGGGACAGTGCTCTGCCCCTGTTCCGGGAGGTACCTCCTTGCTGGC
AAGTGTGTGGAGCTCCCTGACTGTCTAGATCACTTGGGAGACTTCACCTGCGAATGTGCAGT

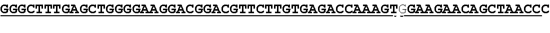

TCGAGGGGACCAAGTTGCCCACCAGGAATGTAACAGCCACTCCAGCAGGTGCTGTGACAAAC
AGAACATGGCCAGGTCAGGTCTATGACAAGCCAGGAGAGATGCCACAGGTCACTGAGATTCT

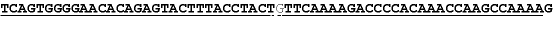

TCACTGGCACACCATCAGGAAGCGTGGTCCTGAACTACACATCTTCGCCCCCTGTTTCTCTG

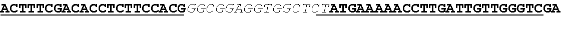

CAACGAAGAAGACATCGATGTTATCCTGAAAAAGTCTACCATTCTGAACTTGGACATCAACA
ACGATATTATCTCCGACATCTCTGGTTTCAACTCCTCTGTTATCACATATCCAGATGCTCAA
TTGGTGCCGGGCATCAACGGCAAAGCTATCCACCTGGTTAACAACGAATCTTCTGAAGTTAT
CGTGCACAAGGCCATGGACATCGAATACAACGACATGTTCAACAACTTCACCGTTAGCTTCT
GGCTGCGCGTTCCGAAAGTTTCTGCTTCCCACCTGGAACAGTACGGCACTAACGAGTACTCC
ATCATCAGCTCTATGAAGAAACACTCCCTGTCCATCGGCTCTGGTTGGTCTGTTTCCCTGAA
GGGTAACAACCTGATCTGGACTCTGAAAGACTCCGCGGGCGAAGTTCGTCAGATCACTTTCC
GCGACCTGCCGGACAAGTTCAACGCGTACCTGGCTAACAAATGGGTTTTCATCACTATCACT
AACGATCGTCTGTCTTCTGCTAACCTGTACATCAACGGCGTTCTGATGGGCTCCGCTGAAAT
CACTGGTCTGGGCGCTATCCGTGAGGACAACAACATCACTCTTAAGCTGGACCGTTGCAACA
ACAACAACCAGTACGTATCCATCGACAAGTTCCGTATCTTCTGCAAAGCACTGAACCCGAAA
GAGATCGAAAAACTGTATACCAGCTACCTGTCTATCACCTTCCTGCGTGACTTCTGGGGTAA
CCCGCTGCGTTACGACACCGAATATTACCTGATCCCGGTAGCTTCTAGCTCTAAAGACGTTC
AGCTGAAAAACATCACTGACTACATGTACCTGACCAACGCGCCGTCCTACACTAACGGTAAA
CTGAACATCTACTACCGACGTCTGTACAACGGCCTGAAATTCATCATCAAACGCTACACTCC
GAACAACGAAATCGATTCTTTCGTTAAATCTGGTGACTTCATCAAACTGTACGTTTCTTACA
ACAACAACGAACACATCGTTGGTTACCCGAAAGACGGTAACGCTTTCAACAACCTGGACAGA
ATTCTGCGTGTTGGTTACAACGCTCCGGGTATCCCGCTGTACAAAAAAATGGAAGCTGTTAA
ACTGCGTGACCTGAAAACCTACTCTGTTCAGCTGAAACTGTACGACGACAAAAACGCTTCTC
TGGGTCTGGTTGGTACCCACAACGGTCAGATCGGTAACGACCCGAACCGTGACATCCTGATC
GCTTCTAACTGGTACTTCAACCACCTGAAAGACAAAATCCTGGGTTGCGACTGGTACTTCGT

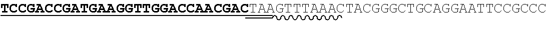

CCCCCCCCCTAACGTTACTGGCCGAAGCCGCTTGGAATAAGGCCGGTGTGCGTTTGTCTATA
TGTTATTTTCCACCATATTGCCGTCTTTTGGCAATGTGAGGGCCCGGAAACCTGGCCCTGTC
TTCTTGACGAGCATTCCTAGGGGTCTTTCCCCTCTCGCCAAAGGAATGCAAGGTCTGTTGAA
TGTCGTGAAGGAAGCAGTTCCTCTGGAAGCTTCTTGAAGACAAACAACGTCTGTAGCGACCC
TTTGCAGGCAGCGGAACCCCCCACCTGGCGACAGGTGCCTCTGCGGCCAAAAGCCACGTGTA
TAAGATACACCTGCAAAGGCGGCACAACCCCAGTGCCACGTTGTGAGTTGGATAGTTGTGGA
AAGAGTCAAATGGCTCTCCTCAAGCGTATTCAACAAGGGGCTGAAGGATGCCCAGAAGGTAC
CCCATTGTATGGGATCTGATCTGGGGCCTCGGTGCACATGCTTTACATGTGTTTAGTCGAGG
TTAAAAAACGTCTAGGCCCCCCGAACCACGGGGACGTGGTTTTCCTTTGAAAAACACGATGA
TAATACCATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCGATGCCACC
TACGGCAAGCTGACCCTGAAGTTCATCTGCACCACCGGCAAGCTGCCCGTGCCCTGGCCCAC
CCTCGTGACCACCCTGACCTACGGCGTGCAGTGCTTCAGCCGCTACCCCGACCACATGAAGC
AGCACGACTTCTTCAAGTCCGCCATGCCCGAAGGCTACGTCCAGGAGCGCACCATCTTCTTC
AAGGACGACGGCAACTACAAGACCCGCGCCGAGGTGAAGTTCGAGGGCGACACCCTGGTGAA
CCGCATCGAGCTGAAGGGCATCGACTTCAAGGAGGACGGCAACATCCTGGGGCACAAGCTGG
AGTACAACTACAACAGCCACAACGTCTATATCATGGCCGACAAGCAGAAGAACGGCATCAAG
GTGAACTTCAAGATCCGCCACAACATCGAGGACGGCAGCGTGCAGCTCGCCGACCACTACCA
GCAGAACACCCCCATCGGCGACGGCCCCGTGCTGCTGCCCGACAACCACTACCTGAGCACCC
AGTCCGCCCTGAGCAAAGACCCCAACGAGAAGCGCGATCACATGGTCCTGCTGGAGTTCGTG
ACCGCCGCCGGGATCACTCTCGGCATGGACGAGCTGTACAAGTCCGGACTCAGATCTCGACT
AGCTAGTAGCTAGCTAGCTAGTCGAGCTCAACTTCGAATTCGATATCAAGCTTATCGCGATA
CCGTCGACCTCGAGGGAATTCCGATAATCAACCTCTGGATTACAAAATTTGTGAAAGATTGA
CTGGTATTCTTAACTATGTTGCTCCTTTTACGCTATGTGGATACGCTGCTTTAATGCCTTTG
TATCATGCTATTGCTTCCCGTATGGCTTTCATTTTCTCCTCCTTGTATAAATCCTGGTTGCT
GTCTCTTTATGAGGAGTTGTGGCCCGTTGTCAGGCAACGTGGCGTGGTGTGCACTGTGTTTG
CTGACGCAACCCCCACTGGTTGGGGCATTGCCACCACCTGTCAGCTCCTTTCCGGGACTTTC
GCTTTCCCCCTCCCTATTGCCACGGCGGAACTCATCGCCGCCTGCCTTGCCCGCTGCTGGAC
AGGGGCTCGGCTGTTGGGCACTGACAATTCCGTGGTGTTGTCGGGGAAGCTGACGTCCTTTC
CATGGCTGCTCGCCTGTGTTGCCACCTGGATTCTGCGCGGGACGTCCTTCTGCTACGTCCCT
TCGGCCCTCAATCCAGCGGACCTTCCTTCCCGCGGCCTGCTGCCGGCTCTGCGGCCTCTTCC
GCGTCTTCGCCTTCGCCCTCAGACGAGTCGGATCTCCCTTTGGGCCGCCTCCCCGCATCGGG
AATTCGAGCTCGGTACCTTTAAGACCAATGACTTACAAGGCAGCTGTAGATCTTAGCCACTT
TTTAAAAGAAAAGGGGGGACTGGAAGGGCTAATTCACTCCCAACGAAGACAAGATGGGATCA
ATTCACCATGGGAATAACTTCGTATAGCATACATTATACGAAGTTATGCTGCTTTTTGCTTG
TACTGGGTCTCTCTGGTTAGACCAGATCTGAGCCTGGGAGCTCTCTGGCTAACTAGGGAACC
CACTGCTTAAGCCTCAATAAAGCTTGCCTTGAGTGCTTCAAGTAGTGTGTGCCCGTCTGTTG
TGTGACTCTGGTAACTAGAGATCCCTCAGACCCTTTTAGTCAGTGTGGAAAATCTCTAGCAG
CATCTAGAATTAATTCCGTGTATTCTATAGTGTCACCTAAATCGTATGTGTATGATACATAA
GGTTATGTATTAATTGTAGCCGCGTTCTAACGACAATATGTACAAGCCTAATTGTGTAGCAT
CTGGCTTACTGAAGCAGACCCTATCATCTCTCTCGTAAACTGCCGTCAGAGTCGGTTTGGTT
GGACGAACCTTCTGAGTTTCTGGTAACGCCGTCCCGCACCCGGAAATGGTCAGCGAACCAAT
CAGCAGGGTCATCGCTAGCCAGATCCTCTACGCCGGACGCATCGTGGCCGGCATCACCGGCG
CCACAGGTGCGGTTGCTGGCGCCTATATCGCCGACATCACCGATGGGGAAGATCGGGCTCGC
CACTTCGGGCTCATGAGCGCTTGTTTCGGCGTGGGTATGGTGGCAGGCCCCGTGGCCGGGGG
ACTGTTGGGCGCCATCTCCTTGCATGCACCATTCCTTGCGGCGGCGGTGCTCAACGGCCTCA
ACCTACTACTGGGCTGCTTCCTAATGCAGGAGTCGCATAAGGGAGAGCGTCGAATGGTGCAC
TCTCAGTACAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGACGTCAG
GTGGCACTTTTCGGGGAAATGTGCGCGGAACCCCTATTTGTTTATTTTTCTAAATACATTCA
AATATGTATCCGCTCATGAGACAATAACCCTGATAAATGCTTCAATAATATTGAAAAAGGAA
GAGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTCGCCCCGA
AGAACGTTTTCCAATGATGAGCACTTTTAAAGTTCTGCTATGTGGCGCGGTATTATCCCGTA
TTGACGCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATGCAGTGC
TGCCATAACCATGAGTGATAACACTGCGGCCAACTTACTTCTGACAACGATCGGAGGACCGA
AGGAGCTAACCGCTTTTTTGCACAACATGGGGGATCATGTAACTCGCCTTGATCGTTGGGAA
CCGGAGCTGAATGAAGCCATACCAAACGACGAGCGTGACACCACGATGCCTGTAGCAATGGC
AACAACGTTGCGCAAACTATTAACTGGCGAACTACTTACTCTAGCTTCCCGGCAACAATTAA
TAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGGCAACTA
TGGATGAACGAAATAGACAGATCGCTGAGATAGGTGCCTCACTGATTAAGCATTGGTAACTG
TCAGACCAAGTTTACTCATATATACTTTAGATTGATTTAAAACTTCATTTTTAATTTAAAAG
GATCTAGGTGAAGATCCTTTTTGATAATCTCATGACCAAAATCCCTTAACGTGAGTTTTCGT
TCCACTGAGCGTCAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGTTTGTTTGCCGGA
TCAAGAGCTACCAACTCTTTTTCCGAAGGTAACTGGCTTCAGCAGAGCGCAGATACCAAATA
CTGTCCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTAC
CGGGTTGGACTCAAGACGATAGTTACCGGATAAGGCGCAGCGGTCGGGCTGAACGGGGGGTT
CGTGCACACAGCCCAGCTTGGAGCGAACGACCTACACCGAACTGAGATACCTACAGCGTGAG
CATTGAGAAAGCGCCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGGTATCTTTATAGTC
CTGTCGGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGGCCTTT
TGCTCACATGTTCTTTCCTGCGTTATCCCCTGATTCTGTGGATAACCGTATTACCGCCTTTG
AGTGAGCTGATACCGCTCGCCGCAGCCGAACGACCGAGCGCAGCGAGTCAGTGAGCGAGGAA
GCGGAAGAGCGCCCAATACGCAAACCGCCTCTCCCCGCGCGTTGGCCGATTCATTAATGCAG
CTGTGGAATGTGTGTCAGTTAGGGTGTGGAAAGTCCCCAGGCTCCCCAGCAGGCAGAAGTAT
GCAAAGCATGCATCTCAATTAGTCAGCAACCAGGTGTGGAAAGTCCCCAGGCTCCCCAGCAG
GCAGAAGTATGCAAAGCATGCATCTCAATTAGTCAGCAACCATAGTCCCGCCCCTAACTCCG
CCCATCCCGCCCCTAACTCCGCCCAGTTCCGCCCATTCTCCGCCCCATGGCTGACTAATTTT
TTTTATTTATGCAGAGGCCGAGGCCGCCTCGGCCTCTGAGCTATTCCAGAAGTAGTGAGGAG
GCTTTTTTGGAGGCCTAGGCTTTTGCAAAAAGCTTGGACACAAGACAGGCTTGCGAGATATG
TTTGAGAATACCACTTTATCCCGCGTCAGGGAGAGGCAGTGCGTAAAAAGACGCGGACTCAT
GTGAAATACTGGTTTTTAGTGCGCCAGATCTCTATAATCTCGCGCAACCTATTTTCCCCTCG
AACACTTTTTAAGCCGTAGATAAACAGGCTGGGACACTTCACATGAGCGAAAAATACATCGT
CACCTGGGACATGTTGCAGATCCATGCACGTAAACTCGCAAGCCGACTGATGCCTTCTGAAC
AATGGAAAGGCATTATTGCCGTAAGCCGTGGCGGTCTGTACCGGGTGCGTTACTGGCGCGTG
AACTGGGTATTCGTCATGTCGATACCGTTTGTATTTCCAGCTACGATCACGACAACCAGCGC
GAGCTTAAAGTGCTGAAACGCGCAGAAGGCGATGGCGAAGGCTTCATCGTTATTGATGACCT
GGTGGATACCGGTGGTACTGCGGTTGCGATTCGTGAAATGTATCCAAAAGCGCACTTTGTCA
CCATCTTCGCAAAACCGGCTGGTCGTCCGCTGGTTGATGACTATGTTGTTGATATCCCGCAA
GATACCTGGATTGAACAGCCGTGGGATATGGGCGTCGTATTCGTCCCGCCAATCTCCGGTCG
CTAATCTTTTCAACGCCTGGCACTGCCGGGCGTTGTTCTTTTTAACTTCAGGCGGGTTACAA
TAGTTTCCAGTAAGTATTCTGGAGGCTGCATCCATGACACAGGCAAACCTGAGCGAAACCCT
GTTCAAACCCCGCTTTAAACATCCTGAAACCTCGACGCTAGTCCGCCGCTTTAATCACGGCG
CACAACCGCCTGTGCAGTCGGCCCTTGATGGTAAAACCATCCCTCACTGGTATCGCATGATT
AACCGTCTGATGTGGATCTGGCGCGGCATTGACCCACGCGAAATCCTCGACGTCCAGGCACG
TATTGTGATGAGCGATGCCGAACGTACCGACGATGATTTATACGATACGGTGATTGGCTACC
GTGGCGGCAACTGGATTTATGAGTGGGCCCCGGATCTTTGTGAAGGAACCTTACTTCTGTGG
TGTGACATAATTGGACAAACTACCTACAGAGATTTAAAGCTCTAAGGTAAATATAAAATTTT
TAAGTGTATAATGTGTTAAACTACTGATTCTAATTGTTTGTGTATTTTAGATTCCAACCTAT
GGAACTGATGAATGGGAGCAGTGGTGGAATGCCTTTAATGAGGAAAACCTGTTTTGCTCAGA
AGAAATGCCATCTAGTGATGATGAGGCTACTGCTGACTCTCAACATTCTACTCCTCCAAAAA
AGAAGAGAAAGGTAGAAGACCCCAAGGACTTTCCTTCAGAATTGCTAAGTTTTTTGAGTCAT
GCTGTGTTTAGTAATAGAACTCTTGCTTGCTTTGCTATTTACACCACAAAGGAAAAAGCTGC
ACTGCTATACAAGAAAATTATGGAAAAATATTCTGTAACCTTTATAAGTAGGCATAACAGTT
ATAATCATAACATACTGTTTTTTCTTACTCCACACAGGCATAGAGTGTCTGCTATTAATAAC
TATGCTCAAAAATTGTGTACCTTTAGCTTTTTAATTTGTAAAGGGGTTAATAAGGAATATTT
GATGTATAGTGCCTTGACTAGAGATCATAATCAGCCATACCACATTTGTAGAGGTTTTACTT
GCTTTAAAAAACCTCCCACACCTCCCCCTGAACCTGAAACATAAAATGAATGCAATTGTTGT
TGTTAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCT
TATCATGTCTGGATCAACTGGATAACTCAAGCTAACCAAAATCATCCCAAACTTCCCACCCC
ATACCCTATTACCACTGCCAATTACCTAGTGGTTTCATTTACTCTAAACCTGTGATTCCTCT
GAATTATTTTCATTTTAAAGAAATTGTATTTGTTAAATATGTACTACAAACTTAGTAG
Key
The sequence that EF-1a (Human elongation factor-1a promoter) Forward Primer
binds
The restriction enzyme sites of PacI and PmeI respectively
The start codon/amino acid methionine
The mouse Clec14a-FrC sequences
The linker sequence glyglyglyglyser between Clec14a and FrC sequences
The stop codon/amino acid
The sequence that IRES Reverse Primer binds
The mutations that were revealed after sequencing
An Amino acid change
Protein sequence of mouse Clec14a-FrC-846 amino acids (pI = 5.99, MW = 93522.86) 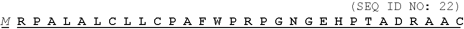 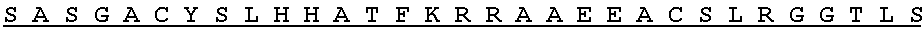 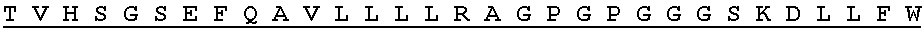 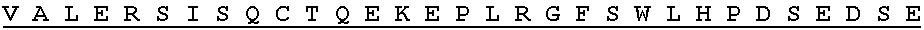 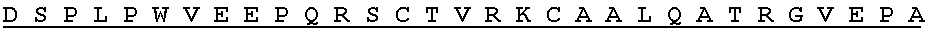 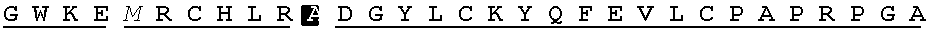 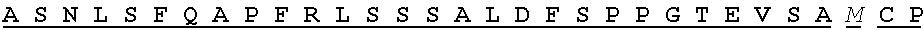 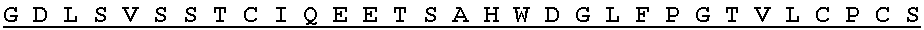 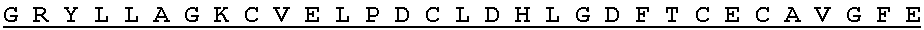 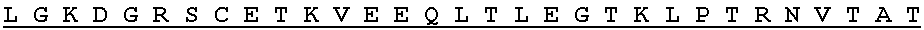 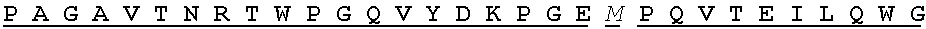 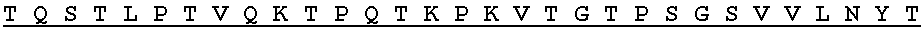 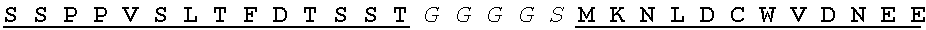 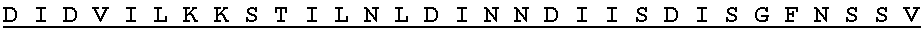 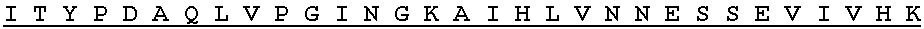 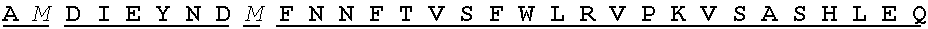 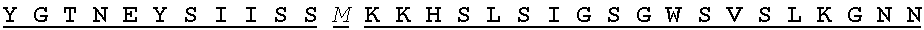 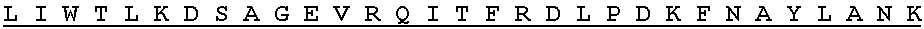 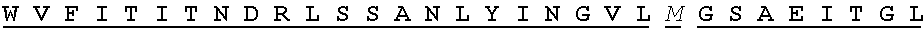 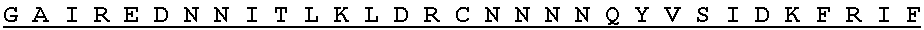 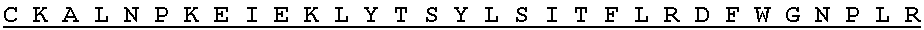 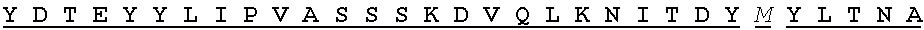 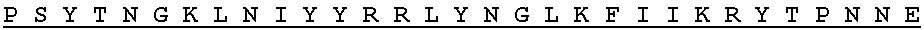 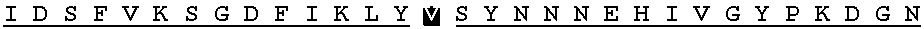 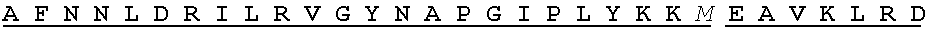 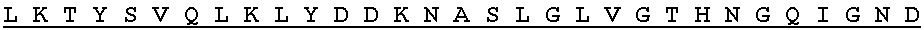 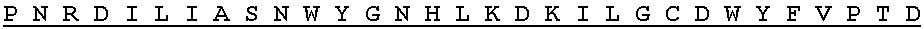 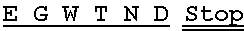
There are 2 amino acid changes (highlighted in black) due to the nucleotide mutations:
1. Threonine (ACT, ACC, ACA, ACG) changed to Alanine (GCT, GCC, GCA, GCG)
2. Isoleucine (ATT, ATC, ATA) changed to Valine (GTT, GTC, GTA, GTG)

### REFERENCES

Attia, M. A., and D. W. Weiss. 1966. Immunology of spontaneous mammary carcinomas in mice. V. Acquired tumor resistance and enhancement in strain A mice infected with mammary tumor virus. Cancer Res 26:1787-1800.
Eskola, J., H. Peltola, A. K. Takala, H. Kayhty, M. Hakulinen, V. Karanko, E. Kela, P. Rekola, P. R. Ronnberg, J. S. Samuelson, and et al. 1987. Efficacy of Haemophilus influenzae type b polysaccharide-diphtheria toxoid conjugate vaccine in infancy. N Engl J Med 317:717-722.
Heath, V. L., and R. Bicknell. 2009. Anticancer strategies involving the vasculature. Nature reviews. Clinical oncology 6:395-404.
Mura, M., R. K. Swain, X. Zhuang, H. Vorschmitt, G. Reynolds, S. Durant, J. F. Beesley, J. M. Herbert, H. Sheldon, M. Andre, S. Sanderson, K. Glen, N. T. Luu, H. M. McGettrick, P. Antczak, F. Falciani, G. B. Nash, Z. S. Nagy, and R. Bicknell. 2012. Identification and angiogenic role of the novel tumor endothelial marker CLEC14A. Oncogene 31:293-305.
Schneerson, R., J. B. Robbins, J. C. Parke, Jr., C. Bell, J. J. Schlesselman, A. Sutton, Z. Wang, G. Schiffman, A. Karpas, and J. Shiloach. 1986. Quantitative and qualitative analyses of serum antibodies elicited in adults by Haemophilus influenzae type b and pneumococcus type 6A capsular polysaccharide-tetanus toxoid conjugates. Infect Immun 52:519-528.

1. A composition for provoking an immune memory response in a patient to an autoantigen target, the composition comprising:
the target conjugated to
a carrier polypeptide, against which immunological memory exists in the patient.

2. A composition according to claim 1, wherein the carrier polypeptide comprises Diphtheria toxoid.

3. A composition according to claim 1, wherein the carrier polypeptide comprises Tetanus toxoid.

4. A composition according to claim 1, wherein the carrier polypeptide comprises an antigen commonly used in human vaccination.

5. A composition according to claim 4, wherein the carrier polypeptide comprises one or more of the antigens used in the polio vaccine.

6. A composition according to claim 4, wherein the carrier polypeptide comprises antigens selected from the group consisting of: measles, mumps, rubella, HPV and pertussis components.

7. A composition according to claim 1, wherein the autoantigen is conjugated to two or more carrier polypeptides.

8. A composition according to claim 7, wherein at least one of the two or more carrier polypeptides is the Diphtheria or the Tetanus toxoid.

9. A composition according to claim 1, comprising a linker to separate the carrier polypeptide from the target.

10. A composition according to claim 1, wherein the target is an autoimmune target, and wherein the autoimmune target is a cancer target against which it is desired to raise a humoral or cell-mediated immune response.

11. A composition according to claim 10, wherein the autoimmune target is Robo4 or Clec14a.

12. A composition according to claim 10, wherein the autoimmune target is, EGFR, Her2, CD38, CD52 or VEGF.

13. A composition according to claim 10 wherein the autoimmune target is CD20 or TN F-alpha.

14. A method for the treatment or prophylaxis of cancer or an autoimmune disease, the method comprising administering to a patient a composition for provoking an immune memory response in a patient to an autoantigen target, the composition comprising:
the target conjugated to
a carrier polypeptide, against which immunological memory exists in the patient.

15. A method according to claim 14, wherein the autoimmune disease is selected from systemic lupus erythematosus, Sjögren's syndrome, scleroderma, rheumatoid arthritis, dermatomyositis, multiple sclerosis, Crohn's disease, psoriasis, psoriatic arthritis, ulcerative colitis and ankylosing spondylitis.

16. A method according to claim 14, wherein the cancer is selected from bladder cancer, pancreatic cancer and lung cancer, including Lewis lung carcinoma.

17. A method of vaccinating an individual and/or boosting a vaccinated individual comprising administering the composition according to claim 1 to a patient to thereby elicit the immune response to said target in said patient.

18. A kit comprising the composition according to claim 1 and a companion diagnostic for a disease condition to be treated in a patient.


SEQUENCE LISTING (22 sequences)

SEQ ID NO: 1 - protein, 386 aa, Corynebacterium diphtheriae
GADDVVDSSKSFVMENFSSYHGTKPGYVDSIQKGIQKPKSGTQGNYDDQWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKT

SEQ ID NO: 2 - protein, 452 aa, Clostridium tetani
MKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 3 - protein, 490 aa, Homo sapiens
  autoantigen
MRPAFALCLLWQALWPGPGGGEHPTADRAGCSASGACYSLHHATMKRQAAEEACILRGGALSTVRAGAELRAVLALLRAGPGPGGGSKDLLFWVALERRRSHCTLENEPLRGFSWLSSDPGGLESDTLQWVEEPQRSCTARRCAVLQATGGVEPAGWKEMRCHLRANGYLCKYQFEVLCPAPRPGAASNLSYRAPFQLHSAALDFSPPGTEVSALCRGQLPISVTCIADEIGARWDKLSGDVLCPCPGRYLRAGKCAELPNCLDDLGGFACECATGFELGKDGRSCVTSGEGQPTLGGTGVPTRRPPATATSPVPQRTWPIRVDEKLGETPLVPEQDNSVTSIPEIPRWGSQSTMSTLQMSLQAESKATITPSGSVISKFNSTTSSATPQAFDSSSAVVFIFVSTAVVVLVILTMTVLGLVKLCFHESPSSQPRKESMGPPGLESDPEPAALGSSSAHCTNNGVKVGDCDLRDRAEGALLAESPLGSSDA

SEQ ID NO: 4 - protein, 1007 aa, Homo sapiens
MGSGGDSLLGGRGSLPLLLLLIMGGMAQDSPPQILVHPQDQLFQGPGPARMSCQASGQPPPTIRWLLNGQPLSMVPPDPHHLLPDGTLLLLQPPARGHAHDGQALSTDLGVYTCEASNRLGTAVSRGARLSVAVLREDFQIQPRDMVAVVGEQFTLECGPPWGHPEPTVSWWKDGKPLALQPGRHTVSGGSLLMARAEKSDEGTYMCVATNSAGHRESRAARVSIQEPQDYTEPVELLAVRIQLENVTLLNPDPAEGPKPRPAVWLSWKVSGPAAPAQSYTALFRTQTAPGGQGAPWAEELLAGWQSAELGGLHWGQDYEFKVRPSSGRARGPDSNVLLLRLPEKVPSAPPQEVTLKPGNGTVFVSWVPPPAENHNGIIRGYQVWSLGNTSLPPANWTVVGEQTQLEIATHMPGSYCVQVAAVTGAGAGEPSRPVCLLLEQAMERATQEPSEHGPWTLEQLRATLKRPEVIATCGVALWLLLLGTAVCIHRRRRARVHLGPGLYRYTSEDAILKHRMDHSDSQWLADTWRSTSGSRDLSSSSSLSSRLGADARDPLDCRRSLLSWDSRSPGVPLLPDTSTFYGSLIAELPSSTPARPSPQVPAVRRLPPQLAQLSSPCSSSDSLCSRRGLSSPRLSLAPAEAWKAKKKQELQHANSSPLLRGSHSLELRACELGNRGSKNLSQSPGAVPQALVAWRALGPKLLSSSNELVTRHLPPAPLFPHETPPTQSQQTQPPVAPQAPSSILLPAAPIPILSPCSPPSPQASSLSGPSPASSRLSSSSLSSLGEDQDSVLTPEEVALCLELSEGEETPRNSVSPMPRAPSPPTTYGYISVPTASEFTDMGRTGGGVGPKGGVLLCPPRPCLTPTPSEGSLANGWGSASEDNAASARASLVSSSDGSFLADAHFARALAVAVDSFGFGLEPREADCVFIDASSPPSPRDEIFLTPNLSLPLWEWRPDWLEDMEVSHTQRLGRGMPPWPPDSQISSQRSQLHCRMPKAGASPVDYS

SEQ ID NO: 5 - protein, 862 aa, Homo sapiens
MVAVVGEQFTLECGPPWGHPEPTVSWWKDGKPLALQPGRHTVSGGSLLMARAEKSDEGTYMCVATNSAGHRESRAARVSIQEPQDYTEPVELLAVRIQLENVTLLNPDPAEGPKPRPAVWLSWKVSGPAAPAQSYTALFRTQTAPGGQGAPWAEELLAGWQSAELGGLHWGQDYEFKVRPSSGRARGPDSNVLLLRLPEKVPSAPPQEVTLKPGNGTVFVSWVPPPAENHNGIIRGYQVWSLGNTSLPPANWTVVGEQTQLEIATHMPGSYCVQVAAVTGAGAGEPSRPVCLLLEQAMERATQEPSEHGPWTLEQLRATLKRPEVIATCGVALWLLLLGTAVCIHRRRRARVHLGPGLYRYTSEDAILKHRMDHSDSQWLADTWRSTSGSRDLSSSSSLSSRLGADARDPLDCRRSLLSWDSRSPGVPLLPDTSTFYGSLIAELPSSTPARPSPQVPAVRRLPPQLAQLSSPCSSSDSLCSRRGLSSPRLSLAPAEAWKAKKKQELQHANSSPLLRGSHSLELRACELGNRGSKNLSQSPGAVPQALVAWRALGPKLLSSSNELVTRHLPPAPLFPHETPPTQSQQTQPPVAPQAPSSILLPAAPIPILSPCSPPSPQASSLSGPSPASSRLSSSSLSSLGEDQDSVLTPEEVALCLELSEGEETPRNSVSPMPRAPSPPTTYGYISVPTASEFTDMGRTGGGVGPKGGVLLCPPRPCLTPTPSEGSLANGWGSASEDNAASARASLVSSSDGSFLADAHFARALAVAVDSFGFGLEPREADCVFIDASSPPSPRDEIFLTPNLSLPLWEWRPDWLEDMEVSHTQRLGRGMPPWPPDSQISSQRSQLHCRMPKAGASPVDYS

SEQ ID NO: 6 - DNA, 40 bp, Artificial Sequence
  primer
ggcggaggtggctctatgaaaaaccttgattgttgggtcg

SEQ ID NO: 7 - DNA, 41 bp, Artificial Sequence
  primer
gagtccgaaagctgaggaggccgcctccaccgagatacttt

SEQ ID NO: 8 - DNA, 38 bp, Artificial Sequence
  primer
tagtaggtttaaacttagtcgttggtccaaccttcatc

SEQ ID NO: 9 - DNA, 33 bp, Artificial Sequence
  primer
tagtagttaattaaaccatgaggccggcgttcg

SEQ ID NO: 10 - DNA, 41 bp, Artificial Sequence
  primer
tttcatagagccacctccgccggaggagtcgaaagcctgag

SEQ ID NO: 11 - DNA, 33 bp, Artificial Sequence; identical to SEQ ID NO: 9
  primer

SEQ ID NO: 12 - DNA, 38 bp, Artificial Sequence; identical to SEQ ID NO: 8
  primer

SEQ ID NO: 13 - DNA, 1436 bp, Clostridium tetani
aagcttgccgccaccatgggttggagctgtatcatcttctttctggtagcaacagctacaggtgtgcactccaaaaaccttgattgttgggtcgacaacgaagaagacatcgatgttatcctgaaaaagtctaccattctgaacttggacatcaacaacgatattatctccgacatctctggtttcaactcctctgttatcacatatccagatgctcaattggtgccgggcatcaacggcaaagctatccacctggttaacaacgaatcttctgaagttatcgtgcacaaggccatggacatcgaatacaacgacatgttcaacaacttcaccgttagcttctggctgcgcgttccgaaagtttctgcttcccacctggaacagtacggcactaacgagtactccatcatcagctctatgaagaaacactccctgtccatcggctctggttggtctgtttccctgaagggtaacaacctgatctggactctgaaagactccgcgggcgaagttcgtcagatcactttccgcgacctgccggacaagttcaacgcgtacctggctaacaaatgggttttcatcactatcactaacgatcgtctgtcttctgctaacctgtacatcaacggcgttctgatgggctccgctgaaatcactggtctgggcgctatccgtgaggacaacaacatcactcttaagctggaccgttgcaacaacaacaaccagtacgtatccatcgacaagttccgtatcttctgcaaagcactgaacccgaaagagatcgaaaaactgtataccagctacctgtctatcaccttcctgcgtgacttctggggtaacccgctgcgttacgacaccgaatattacctgatcccggtagcttctagctctaaagacgttcagctgaaaaacatcactgactacatgtacctgaccaacgcgccgtcctacactaacggtaaactgaacatctactaccgacgtctgtacaacggcctgaaattcatcatcaaacgctacactccgaacaacgaaatcgattctttcgttaaatctggtgacttcatcaaactgtacgtttcttacaacaacaacgaacacatcgttggttacccgaaagacggtaacgctttcaacaacctggacagaattctgcgtgttggttacaacgctccgggtatcccgctgtacaaaaaaatggaagctgttaaactgcgtgacctgaaaacctactctgttcagctgaaactgtacgacgacaaaaacgcttctctgggtctggttggtacccacaacggtcagatcggtaacgacccgaaccgtgacatcctgatcgcttctaactggtacttcaaccacctgaaagacaaaatcctgggttgcgactggtacttcgttccgaccgatgaaggttggaccaacgactaagcggccgc

SEQ ID NO: 14 - DNA, 40 bp, Artificial Sequence; identical to SEQ ID NO: 6
  primer

SEQ ID NO: 15 - DNA, 38 bp, Artificial Sequence; identical to SEQ ID NO: 8
  primer

SEQ ID NO: 16 - DNA, 1908 bp, Homo sapiens
atgaggccggcgttcgccctgtgcctcctctggcaggcgctctggcccgggccgggcggcggcgaacaccccactgccgaccgtgctggctgctcggcctcgggggcctgctacagcctgcaccacgctaccatgaagcggcaggcggccgaggaggcctgcatcctgcgaggtggggcgctcagcaccgtgcgtgcgggcgccgagctgcgcgctgtgctcgcgctcctgcgggcaggcccagggcccggagggggctccaaagacctgctgttctgggtcgcactggagcgcaggcgttcccactgcaccctggagaacgagcctttgcggggtttctcctggctgtcctccgaccccggcggtctcgaaagcgacacgctgcagtgggtggaggagccccaacgctcctgcaccgcgcggagatgcgcggtactccaggccaccggtggggtcgagcccgcaggctggaaggagatgcgatgccacctgcgcgccaacggctacctgtgcaagtaccagtttgaggtcttgtgtcctgcgccgcgccccggggccgcctctaacttgagctatcgcgcgcccttccagctgcacagcgccgctctggacttcagtccacctgggaccgaggtgagtgcgctctgccggggacagctcccgatctcagttacttgcatcgcggacgaaatcggcgctcgctgggacaaactctcgggcgatgtgttgtgtccctgccccgggaggtacctccgtgctggcaaatgcgcagagctccctaactgcctagacgacttgggaggctttgcctgcgaatgtgctacgggcttcgagctggggaaggacggccgctcttgtgtgaccagtggggaaggacagccgacccttggggggaccggggtgcccaccaggcgcccgccggccactgcaaccagccccgtgccgcagagaacatggccaatcagggtcgacgagaagctgggagagacaccacttgtccctgaacaagacaattcagtaacatctattcctgagattcctcgatggggatcacagagcacgatgtctacccttcaaatgtcccttcaagccgagtcaaaggccactatcaccccatcagggagcgtgatttccaagtttaattctacgacttcctctgccactcctcaggctttcgactcctccagcggccgcggttcttcatcgagtgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 17 - DNA, 33 bp, Artificial Sequence; identical to SEQ ID NO: 9
  primer

SEQ ID NO: 18 - DNA, 41 bp, Artificial Sequence; identical to SEQ ID NO: 10
  primer

SEQ ID NO: 19 - DNA, 33 bp, Artificial Sequence; identical to SEQ ID NO: 9
  primer

SEQ ID NO: 20 - DNA, 38 bp, Artificial Sequence; identical to SEQ ID NO: 8
  primer

SEQ ID NO: 21 - DNA, 13450 bp, Mus musculus
ttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatgtcgacgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgacatttaaatttaattaaaccatgaggccagcgcttgccctgtgcctcctctgtcctgcgttctggcctcggccagggaatggggagcatcccacggccgatcgcgcagcttgttcggcctcgggggcttgctacagccttcaccacgctaccttcaagagaagggcggcggaggaggcctgcagcctaaggggcgggactctcagcaccgtgcactcaggctcggagtttcaagctgtgctcctgctcttgcgtgcaggtcccgggcctggcggaggctccaaagatcttctgttctgggtggctctggaacgcagcatctcacagtgcactcaggagaaagagcctttaaggggtttctcctggttgcacccggactcagaagactcagaggacagcccactaccgtgggtggaagagccacaacgttcctgtacagtgagaaagtgcgctgcgctccaggccaccaggggagtggagcctgctggttggaaggagatgcgctgtcatctgcgcgccgatggctacctatgcaagtaccagtttgaggttctgtgccctgcacctcgcccaggagccgcctctaatttgagtttccaagctcccttccggctgagcagctccgcgctggacttcagccctcctgggacagaggtgagtgcgatgtgtcccggggacctctctgtttcgtccacctgcatccaggaagagacaagcgcacactgggacgggcttttccctgggacagtgctctgcccctgttccgggaggtacctccttgctggcaagtgtgtggagctccctgactgtctagatcacttgggagacttcacctgcgaatgtgcagtgggctttgagctggggaaggacggacgttcttgtgagaccaaagtggaagaacagctaaccctcgaggggaccaagttgcccaccaggaatgtaacagccactccagcaggtgctgtgacaaacagaacatggccaggtcaggtctatgacaagccaggagagatgccacaggtcactgagattcttcagtggggaacacagagtactttacctactgttcaaaagaccccacaaaccaagccaaaagtcactggcacaccatcaggaagcgtggtcctgaactacacatcttcgccccctgtttctctgactttcgacacctcttccacgggcggaggtggctctatgaaaaaccttgattgttgggtcgacaacgaagaagacatcgatgttatcctgaaaaagtctaccattctgaacttggacatcaacaacgatattatctccgacatctctggtttcaactcctctgttatcacatatccagatgctcaattggtgccgggcatcaacggcaaagctatccacctggttaacaacgaatcttctgaagttatcgtgcacaaggccatggacatcgaatacaacgacatgttcaacaacttcaccgttagcttctggctgcgcgttccgaaagtttctgcttcccacctggaacagtacggcactaacgagtactccatcatcagctctatgaagaaacactccctgtccatcggctctggttggtctgtttccctgaagggtaacaacctgatctggactctgaaagactccgcgggcgaagttcgtcagatcactttccgcgacctgccggacaagttcaacgcgtacctggctaacaaatgggttttcatcactatcactaacgatcgtctgtcttctgctaacctgtacatcaacggcgttctgatgggctccgctgaaatcactggtctgggcgctatccgtgaggacaacaacatcactcttaagctggaccgttgcaacaacaacaaccagtacgtatccatcgacaagttccgtatcttctgcaaagcactgaacccgaaagagatcgaaaaactgtataccagctacctgtctatcaccttcctgcgtgacttctggggtaacccgctgcgttacgacaccgaatattacctgatcccggtagcttctagctctaaagacgttcagctgaaaaacatcactgactacatgtacctgaccaacgcgccgtcctacactaacggtaaactgaacatctactaccgacgtctgtacaacggcctgaaattcatcatcaaacgctacactccgaacaacgaaatcgattctttcgttaaatctggtgacttcatcaaactgtacgtttcttacaacaacaacgaacacatcgttggttacccgaaagacggtaacgctttcaacaacctggacagaattctgcgtgttggttacaacgctccgggtatcccgctgtacaaaaaaatggaagctgttaaactgcgtgacctgaaaacctactctgttcagctgaaactgtacgacgacaaaaacgcttctctgggtctggttggtacccacaacggtcagatcggtaacgacccgaaccgtgacatcctgatcgcttctaactggtacttcaaccacctgaaagacaaaatcctgggttgcgactggtacttcgttccgaccgatgaaggttggaccaacgactaagtttaaactacgggctgcaggaattccgcccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctagctagctagtcgagctcaacttcgaattcgatatcaagcttatcgcgataccgtcgacctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcgagctcggtacctttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtag

SEQ ID NO: 22 - protein, 843 aa, Mus musculus
  SIGNAL 843: stop codon
MRPALALCLLCPAFWPRPGNGEHPTADRAACSASGACYSLHHATFKRRAAEEACSLRGGTLSTVHSGSEFQAVLLLLRAGPGPGGGSKDLLFWVALERSISQCTQEKEPLRGFSWLHPDSEDSEDSPLPWVEEPQRSCTVRKCAALQATRGVEPAGWKEMRCHLRADGYLCKYQFEVLCPAPRPGAASNLSFQAPFRLSSSALDFSPPGTEVSAMCPGDLSVSSTCIQEETSAHWDGLFPGTVLCPCSGRYLLAGKCVELPDCLDHLGDFTCECAVGFELGKDGRSCETKVEEQLTLEGTKLPTRNVTATPAGAVTNRTWPGQVYDKPGEMPQVTEILQWGTQSTLPTVQKTPQTKPKVTGTPSGSVVLNYTSSPPVSLTFDTSSTGGGGSMKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND